(19) United States
(12) Patent Application Publication
(10) Pub. No.: US 2025/0263579 A1
(43) Pub. Date: Aug. 21, 2025

(54) CURE-ON-DEMAND POLYMERS AND METHODS OF MAKING AND USE THEREOF

(71) Applicant: Board of Supervisors of Louisiana State University and Agricultural and Mechanical College, Baton Rouge, LA (US)

(72) Inventors: John Pojman, Baton Rouge, LA (US); Md Abdullah Al Mahmud, Baton Rouge, LA (US)

(21) Appl. No.: 19/052,417

(22) Filed: Feb. 13, 2025

Related U.S. Application Data
(60) Provisional application No. 63/553,785, filed on Feb. 15, 2024.

(51) Int. Cl. C09D 181/02; C08K 3/36; C08K 7/06; C09D 7/40; C09D 7/61; C12N 9/80

(52) U.S. Cl. CPC C09D 181/02; C09D 7/61; C09D 7/70; C12N 9/80; C12Y 305/01005; C08K 3/36; C08K 7/06

(57) ABSTRACT

The present disclosure relates to method of depositing a coating over a substrate, The present disclosure also relates to a resin composition and a polymerized coating disposed over a substrate.

### CROSS-REFERENCE TO RELATED APPLICATIONS

This application claims the benefit of and priority to U.S. Provisional Application Ser. No. 63/553,785, having the title “CURE-ON-DEMAND POLYMERS AND METHODS OF MAKING AND USE THEREOF” filed on Feb. 15, 2024, the disclosure of which is incorporated herein in by reference in its entirety.

### BACKGROUND

Cure-on-demand technology can be used to combat challenges in the development and application of coatings. Thermoset polymeric coatings require two components to be mixed prior to application. The reaction starts upon mixing, and there is a limited time before the coating hardens, which is called the “pot life”. The amounts of each component must be accurately measured, and the mixing must be performed carefully. Solvents are often used to reduce the viscosity. The solvent, as much as 40% by weight, produced pollutants (VOCs) was it evaporates. Waterborne thermoset coatings also are two parts and require mixing (Gary et al., 2023, CoatingsTech, 34)

### SUMMARY

The present disclosure relates to compositions for cure-on-demand coatings and methods of using the coatings.

In an aspect, the present disclosure provides for a method of depositing a coating over a substrate, the method comprising the steps of: providing a resin composition; applying the resin composition over a substrate; exposing the resin composition to a solution comprising a base catalyst; and polymerizing the resin composition; wherein the resin composition comprises: a monomer comprising at least one reactive functionality, wherein the reactive functionality is an alkenyl group or a heterocyclic group; an initiator comprising at least one thiol functionality, wherein the initiator is capable of being activated using a base catalyst; wherein the resin composition further comprises: about 0.1-10 phr silica; about 5-50 phr milled carbon fiber; and about 0.1-5 phr of at least one source of urease.

In an aspect, the present disclosure provides for a resin composition comprising: a monomer comprising at least one reactive functionality, wherein the reactive functionality is an alkenyl group or a heterocyclic group; an initiator comprising at least one thiol functionality, wherein the initiator is capable of being activated using a base catalyst; wherein the resin composition further comprises: about 0.1-10 phr silica; about 5-50 phr milled carbon fiber; and about 0.1-5 phr of at least one source of urease.

In an aspect, the present disclosure provides for a polymerized coating disposed over a substrate, the coating comprising: a repeat unit formed from a monomer comprising at least one reactive functionality, wherein the reactive functionality is an alkenyl group or a heterocyclic group; a cross-linker formed from an initiator comprising at least one thiol functionality; wherein the polymerized coating further comprises: about 0.1-10 phr silica; about 5-50 phr milled carbon fiber; about 0.1-5 phr of at least one source of urease; about 0.1-5 phr phenyl phosphonic acid; and about 0.1-5 phr substituted or unsubstituted phenol.

### BRIEF DESCRIPTION OF THE DRAWINGS

The following detailed description of embodiments of the present disclosure will be better understood when read in conjunction with the appended drawings. It should be understood that the present disclosure is not limited to the precise arrangements and instrumentalities of the embodiments shown in the drawings.

FIG. 1 depicts a reaction scheme of the hydrolysis of urea in the presence of urease enzyme.

FIG. 2A and FIG. 2B depict experimental pH-time profiles obtained in the urea-urease reaction. Inset: Relative enzyme rate V′ as a function of pH calculated. Initial concentrations are: [CO(NH2)2]0) 5 mM, [urease]0) 1.4 units mL⁻¹. FIG. 2A depicts initial concentrations [CH₃COOH]₀) as (i) 0.2, (ii) 0.58, and (iii) 0.93 mM. FIG. 2B depicts initial concentrations [H₂SO₄]₀) as (i) 0.05, (ii) 0.11, and (iii) 0.18 mM.

FIG. 3 depicts the chemical structures of compounds utilized in present experiments.

FIG. 4 depicts images of the application and curing technique of the formulation.

FIG. 5 depicts infrared (IR) stretches of major thiol-acrylate functional groups and their disappearance after polymerization.

FIG. 6 depicts IR stretching of major thiol-epoxy functional groups and their appearance and disappearance.

FIG. 7A and FIG. 7B depict curing times. FIG. 7A depicts curing time of thiol-acrylate/epoxy coatings using 28-30% NH₃ solution. FIG. 7B depicts curing time of thiol-acrylate/epoxy coatings using in situ ammonia from urea-urease reaction.

FIG. 8A through FIG. 8C depict images of coatings in various compositions. FIG. 8A depicts an image of a coating without fillers after curing. FIG. 8B depicts an image of a coating with fume silica after curing. The full image is 4 in×6 in. FIG. 8C depicts an image of a coating with fillers after curing.

FIG. 9A and FIG. 9B depict images of coating samples after curing on a metal substrate. FIG. 9A depicts a cured coating sample of thiol-epoxy after mechanical testing. FIG. 9B depicts a cured coating sample of thiol-acrylate after mechanical testing.

FIG. 10A and FIG. 10B depict images of coating samples after impact testing. FIG. 10A depicts a BADGE-TT1 containing coating 3′×3′ substrate before impact testing. FIG. 10B depicts a BADGE-TT1 containing coating 3′×3′ substrate after impact testing.

FIG. 11 depicts pot life experiments of coating formulations with TT1 (thiol).

### DETAILED DESCRIPTION

The present disclosure relates to compositions for cure-on-demand coatings and methods of using the coatings.

This disclosure is not limited to particular embodiments described, and as such may, of course, vary. The terminology used herein serves the purpose of describing particular embodiments only, and is not intended to be limiting, since the scope of the present disclosure will be limited only by the appended claims.

Where a range of values is provided, each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method may be carried out in the order of events recited or in any other order that is logically possible.

Embodiments of the present disclosure will employ, unless otherwise indicated, techniques of, chemistry, polymer chemistry, polymer science, and the like, which are within the skill of the art. Such techniques are explained fully in the literature.

Unless otherwise defined, all technical and scientific terms used herein have the same meaning as commonly understood by one of ordinary skill in the art of material science, chemistry, textiles, polymer chemistry, and the like. Although methods and materials similar or equivalent to those described herein can be used in the practice or testing of the present disclosure, suitable methods and materials are described herein.

As used in the specification and the appended claims, the singular forms “a,” “an,” and “the” may include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

### Definitions

Unless defined otherwise, all technical and scientific terms used herein have the same meaning as commonly understood by one of ordinary skill in the art to which the present disclosure belongs.

As used herein, each of the following terms has the meaning associated with it in this section.

As used herein, the term “alkyl,” by itself or as part of another substituent means, unless otherwise stated, a straight or branched chain hydrocarbon having the number of carbon atoms designated (i.e. C₁₋₆ means one to six carbon atoms) and including straight, branched chain, or cyclic substituent groups. Examples include methyl, ethyl, propyl, isopropyl, butyl, isobutyl, tert-butyl, pentyl, neopentyl, hexyl, and cyclopropylmethyl.

As used herein, the term “substituted alkyl” means alkyl as defined above, substituted by one, two or three substituents selected from the group consisting of halogen, —OH, alkoxy, —NH₂, amino, azido, —N(CH₃)₂, —C(═O)OH, trifluoromethyl, —C≡N, —C(═O)O(C₁-C₄)alkyl, —C(═O)NH₂, —SO₂NH₂, —C(═NH)NH₂, and —NO₂. Examples of substituted alkyls include, but are not limited to, 2,2-difluoropropyl, 2-carboxycyclopentyl and 3-chloropropyl.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. In an embodiment, an example can include: —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers.

As used herein, the term “halo” or “halogen” alone or as part of another substituent means, unless otherwise stated, a fluorine, chlorine, bromine, or iodine atom.

As used herein, the term “cycloalkyl” refers to a mono cyclic or polycyclic non-aromatic radical, wherein each of the atoms forming the ring (i.e. skeletal atoms) is a carbon atom. In one embodiment, the cycloalkyl group is saturated or partially unsaturated. In another embodiment, the cycloalkyl group is fused with an aromatic ring. Cycloalkyl groups include groups having from 3 to 10 ring atoms. Illustrative examples of cycloalkyl groups include, but are not limited to, the following moieties:

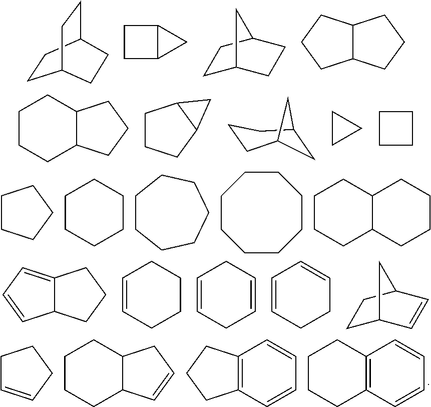

Monocyclic cycloalkyls include, but are not limited to, cyclopropyl, cyclobutyl, cyclopentyl, cyclohexyl, cycloheptyl, and cyclooctyl. Dicyclic cycloalkyls include, but are not limited to, tetrahydronaphthyl, indanyl, and tetrahydropentalene. Polycyclic cycloalkyls include adamantine and norbornane. The term cycloalkyl includes “unsaturated nonaromatic carbocyclyl” or “nonaromatic unsaturated carbocyclyl” groups, both of which refer to a nonaromatic carbocycle as defined herein, which contains at least one carbon double bond or one carbon triple bond.

As used herein, the term “heterocycloalkyl” or “heterocyclyl” or “heterocyclic” refers to a cyclic group containing one to four ring heteroatoms each selected from O, S, and N. In one embodiment, each heterocycloalkyl group has from 4 to 10 atoms in its ring system, with the proviso that the ring of said group does not contain two adjacent O atoms. In another embodiment, the heterocycloalkyl group is fused with an aromatic ring. In one embodiment, the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In one embodiment, the heterocycle is a heteroaryl.

An example of a 3-membered heterocycloalkyl group includes, and is not limited to, aziridine. Examples of 4-membered heterocycloalkyl groups include, and are not limited to, azetidine and a beta lactam. Examples of 5-membered heterocycloalkyl groups include, and are not limited to, pyrrolidine, oxazolidine and thiazolidinedione. Examples of 6-membered heterocycloalkyl groups include, and are not limited to, piperidine, morpholine and piperazine. Other non-limiting examples of heterocycloalkyl groups are:

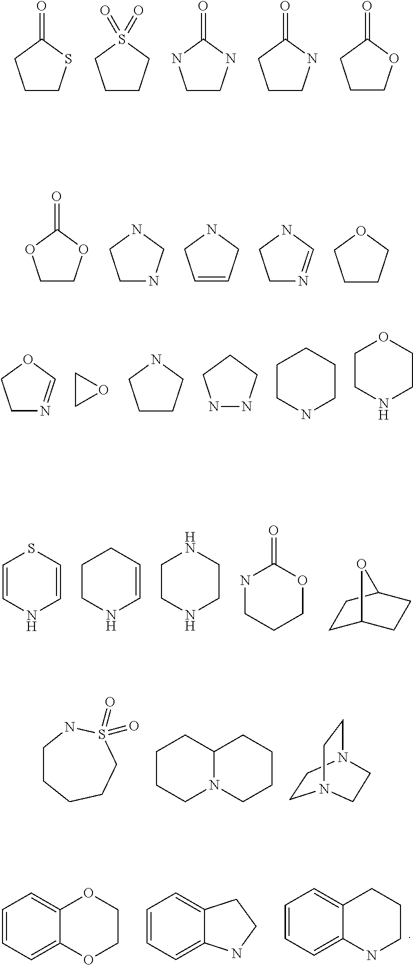

Examples of non-aromatic heterocycles include monocyclic groups such as aziridine, oxirane, thiirane, azetidine, oxetane, thietane, pyrrolidine, pyrroline, pyrazolidine, imidazoline, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin, and hexamethyleneoxide.

As used herein, the term “aromatic” refers to a carbocycle or heterocycle with one or more polyunsaturated rings and having aromatic character, i.e. having (4n+2) delocalized π (pi) electrons, where n is an integer.

As used herein, the term “aryl,” employed alone or in combination with other terms, means, unless otherwise stated, a carbocyclic aromatic system containing one or more rings (typically one, two or three rings), wherein such rings may be attached together in a pendent manner, such as a biphenyl, or may be fused, such as naphthalene. Examples of aryl groups include phenyl, anthracyl, and naphthyl.

As used herein, the term “aryl-(C₁-C₃)alkyl” means a functional group wherein a one- to three-carbon alkylene chain is attached to an aryl group, e.g., —CH₂CH₂-phenyl, —CH₂-phenyl (benzyl), aryl-CH₂— and aryl-CH(CH₃)—. The term “substituted aryl-(C₁-C₃)alkyl” means an aryl-(C₁-C₃)alkyl functional group in which the aryl group is substituted. Similarly, the term “heteroaryl-(C₁-C₃)alkyl” means a functional group wherein a one to three carbon alkylene chain is attached to a heteroaryl group, e.g., —CH₂CH₂-pyridyl. The term “substituted heteroaryl-(C₁-C₃)alkyl” means a heteroaryl-(C₁-C₃)alkyl functional group in which the heteroaryl group is substituted.

As used herein, the term “heteroaryl” or “heteroaromatic” refers to a heterocycle having aromatic character. A polycyclic heteroaryl may include one or more rings that are partially saturated. Examples include the following moieties:

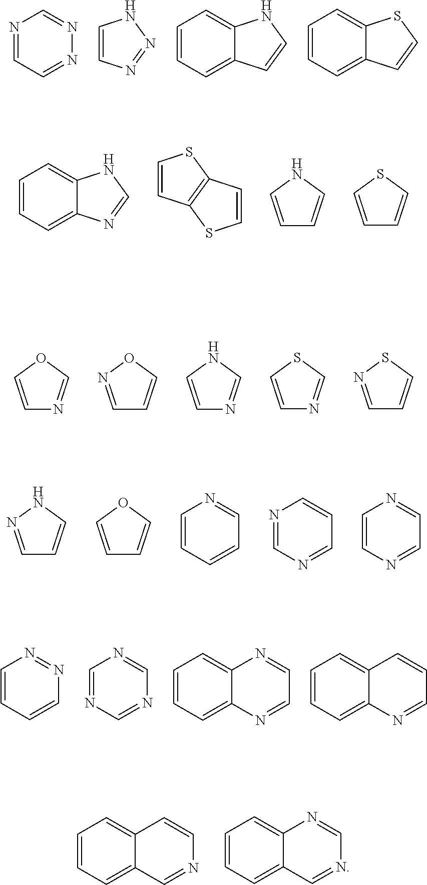

Examples of heteroaryl groups also include pyridyl, pyrazinyl, pyrimidinyl (particularly 2- and 4-pyrimidinyl), pyridazinyl, thienyl, furyl, pyrrolyl (particularly 2-pyrrolyl), imidazolyl, thiazolyl, oxazolyl, pyrazolyl (particularly 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles and heteroaryls include indolyl (particularly 3-, 4-, 5-, 6- and 7-indolyl), indolinyl, quinolyl, tetrahydroquinolyl, isoquinolyl (particularly 1- and 5-isoquinolyl), 1,2,3,4-tetrahydroisoquinolyl, cinnolinyl, quinoxalinyl (particularly 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (particularly 3-, 4-, 5-, 6- and 7-benzofuryl), 2,3-dihydrobenzofuryl, 1,2-benzisoxazolyl, benzothienyl (particularly 3-, 4-, 5-, 6-, and 7-benzothienyl), benzoxazolyl, benzothiazolyl (particularly 2-benzothiazolyl and 5-benzothiazolyl), purinyl, benzimidazolyl (particularly 2-benzimidazolyl), benzotriazolyl, thioxanthinyl, carbazolyl, carbolinyl, acridinyl, pyrrolizidinyl, and quinolizidinyl.

As used herein, the term “substituted” means that an atom or group of atoms has replaced hydrogen as the substituent attached to another group. The term “substituted” further refers to any level of substitution, namely mono-, di-, tri-, tetra-, or penta-substitution, where such substitution is permitted. The substituents are independently selected, and substitution may be at any chemically accessible position. In one embodiment, the substituents vary in number between one and four. In another embodiment, the substituents vary in number between one and three. In yet another embodiment, the substituents vary in number between one and two.

As used herein, the term “optionally substituted” means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

In one embodiment, the substituents are independently selected from the group consisting of oxo, halogen, —CN, —NH₂, —OH, —NH(CH₃), —N(CH₃)₂, alkyl (including straight chain, branched and/or unsaturated alkyl), substituted or unsubstituted cycloalkyl, substituted or unsubstituted heterocycloalkyl, fluoro alkyl, substituted or unsubstituted heteroalkyl, substituted or unsubstituted alkoxy, fluoroalkoxy, —S-alkyl, S(═O)₂alkyl, S(═O)₂N[H, alkyl, or aryl], —C(═O)NH[substituted or unsubstituted alkyl, or substituted or unsubstituted phenyl], —C(═O)N[H or substituted or unsubstituted alkyl or aryl]₂, —OC(═O)N[substituted or unsubstituted alkyl]₂, —NHC(═O)NH[substituted or unsubstituted alkyl, or substituted or unsubstituted phenyl], —NHC(═O)alkyl, —N[substituted or unsubstituted alkyl]C(═O)[substituted or unsubstituted alkyl], —NHC(═O)[substituted or unsubstituted alkyl], —C(OH)[substituted or unsubstituted alkyl]₂, and —C(NH₂)[substituted or unsubstituted alkyl]₂. In another embodiment, by way of example, an optional substituent is selected from oxo, fluorine, chlorine, bromine, iodine, —CN, —NH₂, —OH, —NH(CH₃), —N(CH₃)₂, —CH₃, —CH₂CH₃, —CH(CH₃)₂, —CF₃, —CH₂CF₃, —OCH₃, —OCH₂CH₃, —OCH(CH₃)₂, —OCF₃, —OCH₂CF₃, —S(═O)₂—CH₃, —C(═O)NH₂, —C(═O)—NHCH₃, —NHC(═O)NHCH₃, —C(═O)CH₃, —ON(O)₂, and —C(═O)OH. In yet one embodiment, the substituents are independently selected from the group consisting of C₁₋₆ alkyl, —OH, C₁₋₆ alkoxy, halo, amino, acetamido, oxo and nitro. As used herein, where a substituent is an alkyl or alkoxy group, the carbon chain may be branched, straight or cyclic.

Several references to integers and R, R¹, R², R³, R⁴, R⁵, R⁶, etc. are made in chemical structures and moieties disclosed and described herein. Any description of integers and R, R¹, R², R³, R⁴, R⁵, R⁶, etc. in the specification is applicable to any structure or moiety reciting integers and R, R¹, R², R³, R⁴, R⁵, R⁶, etc. respectively.

As used herein, “phr” refers to parts per hundred parts rubber or parts per hundred parts resin, where the resin is the sum amount of monomer and initiator.

### Discussion:

The present disclosure provides for resin compositions, coatings including the resin composition, methods of depositing a coating over a substrate, polymerized coatings disposed over a substrate, and the like. In an aspect, the present disclosure can provide one or more of the following advantages: a one-pot formulation that requires no mixing; 100% solids with zero volatile organic compounds (VOCs); after the resin has been applied, it does not react until a layer of ammonia solution or urea is applied; and the beginning of the reaction can be controlled by adjusting the pH of the urea solution. Additional details are provided in the Example.

In one aspect, the present disclosure relates to a method of depositing a coating over a substrate, the method comprising the steps of: providing a resin composition; applying the resin composition over a substrate; exposing the resin composition to a solution comprising a base catalyst; and polymerizing the resin composition; where the resin composition comprises: a monomer comprising at least one reactive functionality, where the reactive functionality is an alkenyl group or a heterocyclic group; an initiator comprising at least one thiol functionality, where the initiator is capable of being activated using a base catalyst; where the resin composition further comprises: about 0-10 or about 0.1-10 phr silica; about 5-50 phr milled carbon fiber; and about 0-5 or about 0.1-5 phr of at least one source of urease.

In one embodiment, exemplary substrates include, but are not limited to, wood, synthetic plastics, metal, and the like. In one embodiment, the substrate is a flat surface. In one embodiment, the substrate is curved. In one embodiment, the substrate is double-sided. In one embodiment, the resin composition is applied to a section of surface the substrate. In one embodiment, the resin composition is applied to the entirety of the surface of the substrate. In one embodiment, the resin composition is applied to both sides of a double-sided substrate. In one embodiment, the substrate is treated prior to application of the resin composition. Exemplary treatments of the substrate include, but are not limited to, chemical treatments (i.e. use of acid, base, etc.) and mechanical treatments (i.e. sanding, polishing, grinding, etc.). In one embodiment, the substrate comprises steel. In one embodiment, the substrate is the steel of a marine vessel. In one embodiment, the substrate is the steel of a metal storage tank. In one embodiment, the substrate is any steel which may be exposed to harsh conditions, such as seawater and caustic chemicals.

In one embodiment, the resin composition is an aqueous composition comprising water. In one embodiment, the resin composition is an organic composition comprising at least one organic solvent. Exemplary organic solvents include, but are not limited to, acetic acid, acetone, acetonitrile, alkanes (e.g., hexanes, heptane), amyl acetate, butanol, butyl acetate, chlorobenzene, chloroform, cumene, cyclohexane, 1,2-dichloroethene, dichloromethane, diethyl ether, dimethoxyethane, dimethylacetamide, dimethylformamide, dimethyl sulfoxide, 1,4-dioxane, ethanol, 2-ethoxyethanol, ethyl acetate, ethyl nitrate, ethyleneglycol, formic acid, hydrazine, isopropanol, methanol, methyl acetate, 2-methyl-1-butanol, 2-methyl-1-propanol, methylbutyl ketone, methylcyclohexane, methylethyl ketone, methylpyrrolidone, methyl tert-butyl ether, nitromethane, propanol, propyl acetate, sulfolane, sarcosine, tetrahydrofuran, tetralin, toluene, 1,1,2-tricholoroethane, triethylamine, urea, xylene, and any combination thereof. In one embodiment, the resin composition is neat (i.e., comprising no additional solvent). In one embodiment, the resin composition comprises a liquid base catalyst.

In one embodiment, the step of exposing the resin composition to a solution comprising a base catalyst is performed by spraying the composition with the solution. In one embodiment, the step is performed by pouring the solution onto the composition. In one embodiment, the step of exposing the resin composition to a solution comprising a base catalyst is performed manually. In one embodiment, the step of exposing the resin composition to a solution comprising a base catalyst is automated. In one embodiment, the step of exposing the resin composition to a solution comprising a base catalyst is performed all at once.

In one embodiment, the method further comprises the step of covering the resin composition. In one embodiment, aluminum foil is used to cover the resin. In one embodiment, plastic is used to cover the resin. In one embodiment, a non-permeable material is used to cover the resin. In one embodiment, the resin is partially covered. In one embodiment, the covering prevents evaporation of the base catalyst or reaction product thereof.

In one embodiment, the step of exposing the resin composition to a solution comprising a base catalyst or reaction product thereof is performed over time. In one embodiment, the resin composition is exposed to the solution for about 1 minute to about 60 hours. In one embodiment, the resin composition is exposed to the solution for about 5 minutes to about 60 hours. In one embodiment, the resin composition is exposed to the solution for about 30 minutes to about 60 hours. In one embodiment, the resin composition is exposed to the solution for about 1 hour to about 60 hours. In one embodiment, the resin composition is exposed to the solution for about 2 hours to about 60 hours. In one embodiment, the resin composition is exposed to the solution for about 3 hours to about 60 hours. In one embodiment, the resin composition is exposed to the solution for about 4 hours to about 60 hours. In one embodiment, the resin composition is exposed to the solution for about 5 hours to about 60 hours. In one embodiment, the resin composition is exposed to the solution for about 6 hours to about 60 hours. In one embodiment, the resin composition is exposed to the solution for about 7 hours to about 60 hours. In one embodiment, the resin composition is exposed to the solution for about 8 hours to about 60 hours. In one embodiment, the resin composition is exposed to the solution for about 9 hours to about 60 hours. In one embodiment, the resin composition is exposed to the solution for about 10 hours to about 60 hours. In one embodiment, the resin composition is exposed to the solution for about 11 hours to about 60 hours. In one embodiment, the resin composition is exposed to the solution for about 12 hours to about 60 hours. In one embodiment, the resin composition is exposed to the solution for about 13 hours to about 60 hours. In one embodiment, the resin composition is exposed to the solution for about 14 hours to about 60 hours. In one embodiment, the resin composition is exposed to the solution for about 15 hours to about 60 hours. In one embodiment, the resin composition is exposed to the solution for about 16 hours to about 60 hours. In one embodiment, the resin composition is exposed to the solution for about 17 hours to about 60 hours. In one embodiment, the resin composition is exposed to the solution for about 18 hours to about 60 hours. In one embodiment, the resin composition is exposed to the solution for about 19 hours to about 60 hours. In one embodiment, the resin composition is exposed to the solution for about 20 hours to about 60 hours. In one embodiment, the resin composition is exposed to the solution for about 21 hours to about 60 hours. In one embodiment, the resin composition is exposed to the solution for about 22 hours to about 60 hours. In one embodiment, the resin composition is exposed to the solution for about 23 hours to about 60 hours. In one embodiment, the resin composition is exposed to the solution for about 24 hours to about 60 hours. In one embodiment, the resin composition is exposed to the solution for about 24 hours to about 36 hours. In one embodiment, the resin composition is exposed to the solution for about 36 hours to about 60 hours. In one embodiment, the resin composition is exposed to the solution for about 21 hours to about 48 hours. In one embodiment, the resin composition is exposed to the solution for about 48 hours to about 60 hours.

In one embodiment, the solution comprising a base catalyst comprises at least one compound selected from the group consisting of ammonia, urea, substituted or unsubstituted amine, substituted or unsubstituted hydroxylamine, and combinations thereof. In one embodiment, the base catalyst is inorganic. In one embodiment, the base catalyst is organic. In one embodiment, the base catalyst diffuses readily into the resin composition. In one embodiment, the base catalyst is not volatile. In one embodiment, the base-catalyst is non-toxic. In one embodiment, the base catalyst is bio-sourced. In one embodiment, the base-catalyst is biocompatible. In one embodiment, the solution comprises urea. In one embodiment, the solution comprises ammonia.

In one embodiment, the solution comprising a base catalyst is aqueous. In one embodiment, the solution comprising a base catalyst comprises an organic solvent. In one embodiment, the solution comprising a base catalyst comprises an inorganic solvent such as ammonia or its derivatives. In one embodiment, the solution comprising a base catalyst is about 1% to about 50% base. In one embodiment, the solution comprising a base catalyst is about 2% to about 50% base. In one embodiment, the solution comprising a base catalyst is about 3% to about 50% base. In one embodiment, the solution comprising a base catalyst is about 4% to about 50% base. In one embodiment, the solution comprising a base catalyst is about 5% to about 50% base. In one embodiment, the solution comprising a base catalyst is about 6% to about 50% base. In one embodiment, the solution comprising a base catalyst is about 7% to about 50% base. In one embodiment, the solution comprising a base catalyst is about 8% to about 50% base. In one embodiment, the solution comprising a base catalyst is about 9% to about 50% base. In one embodiment, the solution comprising a base catalyst is about 10% to about 50% base. In one embodiment, the solution comprising a base catalyst is about 11% to about 50% base. In one embodiment, the solution comprising a base catalyst is about 12% to about 50% base. In one embodiment, the solution comprising a base catalyst is about 13% to about 50% base. In one embodiment, the solution comprising a base catalyst is about 14% to about 50% base. In one embodiment, the solution comprising a base catalyst is about 15% to about 50% base. In one embodiment, the solution comprising a base catalyst is about 20% to about 50% base. In one embodiment, the solution comprising a base catalyst is about 30% to about 50% base. In one embodiment, the solution comprising a base catalyst is about 40% to about 50% base. In one embodiment, the solution comprising a base catalyst is about 1% to about 30% base. In one embodiment, the solution comprising a base catalyst is about 2% to about 30% base. In one embodiment, the solution comprising a base catalyst is about 3% to about 30% base. In one embodiment, the solution comprising a base catalyst is about 4% to about 30% base. In one embodiment, the solution comprising a base catalyst is about 5% to about 30% base. In one embodiment, the solution comprising a base catalyst is about 6% to about 30% base. In one embodiment, the solution comprising a base catalyst is about 7% to about 30% base. In one embodiment, the solution comprising a base catalyst is about 8% to about 30% base. In one embodiment, the solution comprising a base catalyst is about 9% to about 30% base. In one embodiment, the solution comprising a base catalyst is about 10% to about 30% base. In one embodiment, the solution comprising a base catalyst is about 11% to about 30% base. In one embodiment, the solution comprising a base catalyst is about 12% to about 30% base. In one embodiment, the solution comprising a base catalyst is about 13% to about 30% base. In one embodiment, the solution comprising a base catalyst is about 14% to about 30% base. In one embodiment, the solution comprising a base catalyst is about 15% to about 30% base. In one embodiment, the solution comprising a base catalyst is about 1% to about 10% base. In one embodiment, the solution comprising a base catalyst is about 2% to about 10% base. In one embodiment, the solution comprising a base catalyst is about 3% to about 10% base. In one embodiment, the solution comprising a base catalyst is about 4% to about 10% base. In one embodiment, the solution comprising a base catalyst is about 5% to about 10% base. In one embodiment, the solution comprising a base catalyst is about 6% to about 10% base. In one embodiment, the solution comprising a base catalyst is about 7% to about 10% base. In one embodiment, the solution comprising a base catalyst is about 8% to about 10% base. In one embodiment, the solution comprising a base catalyst is about 9% to about 10% base. In one embodiment, the base is ammonia. In one embodiment, the base is urea.

In one embodiment, the solution comprising a base catalyst is further acidified using an acid. Exemplary acids include, but are not limited to, organic acids (i.e. acetic acid, citric acid, formic acid, itaconic acid, etc.) and inorganic acids (i.e. carbonic acid, phosphoric acid, hydrogen chloride, etc.). In one embodiment, the solution is slightly acidic.

As used herein, “urease” (e.g., urea amidohydrolase, EC 3.5.1.5) catalyzes the hydrolysis of urea, such as to yield ammonia ions and bicarbonate ions. In one embodiment, urease is a multimer protein composed of several 90 kDa monomers. For the present disclosure, the urease source can be a natural or synthetic source of urease, or both. “Natural sources of urease,” as used herein, refers to any portion of a living or dead organism, including plants, bacteria, animal, algae, or fungi from which urease may be extracted or otherwise separated. Canavalia ensiformis (“Jack beans”) is a natural source of urease. Other natural sources of urease include, but are not limited to, soy beans, watermelon seeds, sword beans (Canavalia gladiate), bacteria (e.g., H. pylori bacteria, Bacillus pasteurii bacterial), or other plants, algae, bacteria or fungi, and/or invertebrate sources of urease (e.g., ruminal urease). Synthetic urease can be recombinant urease, such as Helicobacter pylori recombinant urease B, recombinant Bacillus subtilis urease, recombinant Bacillus pasteurii urease, or other recombinant ureases. Any source of urease or combination of sources of ureases can be used. In one embodiment, the at least one source of urease is selected from the group consisting of watermelon seeds, jack beans, pea seeds, soybeans, sword beans, ruminal urease, and synthetic urease. In one embodiment, the at least one source of urease is watermelon seeds. In one embodiment, the at least one source of urease is dried. In one embodiment, the at least one source of urease is liquid. In one embodiment, the at least one source of urease is solid. In one embodiment, the at least the one source of urease is ground, milled, crushed, macerated, or a combination thereof.

In one embodiment, the molar ratio of base to urease is about 1:30. In one embodiment, the molar ratio is about 1:20. In one embodiment, the molar ratio is about 3:10. In one embodiment, the molar ratio is about 3:20. In one embodiment, the molar ratio is about 3:40. In one embodiment, the molar ratio is about 3:50. In one embodiment, the molar ratio is about 3:70. In one embodiment, the molar ratio is about 3:100. In one embodiment, the base is urea.

In one embodiment, the at least one source of urease is watermelon seed or watermelon seed particles. In one embodiment, the concentration of the urease is modulated by altering the size of the watermelon seed particles.

In one embodiment, the method displays temporal control of the induction time in the polymerization. In one embodiment, the polymerization step occurs immediately after the resin composition is exposed to a solution comprising a base catalyst. In one embodiment, the polymerization step occurs about 30 seconds after the resin composition is exposed to a solution comprising a base catalyst. In one embodiment, the polymerization step occurs about 1 minute after the resin composition is exposed to a solution comprising a base catalyst. In one embodiment, the polymerization step occurs about 5 minutes after the resin composition is exposed to a solution comprising a base catalyst. In one embodiment, the polymerization step occurs about 10 minutes after the resin composition is exposed to a solution comprising a base catalyst. In one embodiment, the polymerization step occurs about 30 minutes after the resin composition is exposed to a solution comprising a base catalyst. In one embodiment, the polymerization step occurs about 1 hour after the resin composition is exposed to a solution comprising a base catalyst. In one embodiment, the polymerization step occurs about 2 hours after the resin composition is exposed to a solution comprising a base catalyst. In one embodiment, the polymerization step occurs about 3 hours after the resin composition is exposed to a solution comprising a base catalyst. In one embodiment, the polymerization step occurs about 4 hours after the resin composition is exposed to a solution comprising a base catalyst. In one embodiment, the polymerization step occurs about 5 hours after the resin composition is exposed to a solution comprising a base catalyst. In one embodiment, the polymerization step occurs about 6 hours after the resin composition is exposed to a solution comprising a base catalyst. In one embodiment, the polymerization step occurs about 12 hours after the resin composition is exposed to a solution comprising a base catalyst. In one embodiment, the polymerization step occurs about 24 hours after the resin composition is exposed to a solution comprising a base catalyst. In one embodiment, the polymerization step occurs about 30 hours after the resin composition is exposed to a solution comprising a base catalyst. In one embodiment, the polymerization step occurs about 36 hours after the resin composition is exposed to a solution comprising a base catalyst. In one embodiment, the polymerization step occurs about 42 hours after the resin composition is exposed to a solution comprising a base catalyst. In one embodiment, the polymerization step occurs about 48 hours after the resin composition is exposed to a solution comprising a base catalyst.

Now having described methods of the present disclosure, additional features are provided. In one aspect, the present disclosure relates to a resin composition comprising a monomer comprising at least one reactive functionality, wherein the reactive functionality is an alkenyl group or a heterocyclic group; an initiator comprising at least one thiol functionality, wherein the initiator is capable of being activated using a base catalyst; wherein the resin composition further comprises: about 0-10 or about 0.1-10 phr silica; about 5-50 phr milled carbon fiber; and about 0-5 or 0.1-5 phr of at least one source of urease.

In one embodiment, the monomer further comprises a moiety selected from the group consisting of bisphenol, glycol, alcohol, quaternary carbon, derivatives thereof, and combinations thereof. In one embodiment, the monomer is selected from the group consisting of an acrylate, a diacrylate, a triacrylate, an epoxy acrylate, an epoxy diacrylate, an epoxy triacrylate, a glycidyl ether, a diglycidyl ether, and a triglycidyl ether, and combinations thereof.

In one embodiment, the monomer comprises at least two reactive functionalities. In one embodiment, the monomer comprises at least three reactive functionalities.

In one embodiment, the reactive functionality is selected from the group consisting of a vinyl group, an acrylate, an epoxide, an oxetane, and an aziridine. In one embodiment, the monomer also serves as a cross-linker.

In one embodiment, the monomer comprises an epoxide. Exemplary such monomers include, but are not limited to, glycidyl acrylate, glycidyl methacrylate (GMA), (E)-(oxiran-2-yl)methyl but-2-enoate, (3,3-dimethyloxiran-2-yl)methyl methacrylate, (E)-(oxiran-2-yl)methyl cinnamate, (oxiran-2-yl)methyl 2-methylenebutanoate, 1-(oxiran-2-yl)propyl acrylate, 1-(oxiran-2-yl)ethyl methacrylate, (oxiran-2-yl)methyl 3-methyl-2-methylenebutanoate, (oxiran-2-yl)methyl 2-methylenepentanoate, (3-methyloxiran-2-yl)methyl acrylate, 2-(oxiran-2-yl)propan-2-yl acrylate, (oxiran-2-yl)methyl 2-methylenehexanoate, (3-methyloxiran-2-yl)methyl methacrylate, and (3,3-dimethyloxiran-2-yl)methyl acrylate, and combinations thereof.

In one embodiment, the monomer comprises an acrylate. Exemplary such monomers include, but are not limited to, esters of acrylic or methacrylic acid with non-tertiary alcohols such as ethanol, 1-propanol, 2-propanol, 1-butanol, 2-butanol, 1-pentanol, 2-pentanol, 3-pentanol, 2-methyl-1-butanol, 3-methyl-1-butanol, 1-hexanol, 2-methyl-1-pentanol, 3-methyl-1-pentanol, 2-ethyl-1-butanol, 3,5, 5-trimethyl-1-hexanol, 3-heptanol, 1-octanol, 2-octanol, isooctanol, 2-ethyl-1-hexanol, 1-decanol, 2-propylheptanol, 1-dodecanol, 1-tridecanol, 1-tetradecanol, citronellol, dihydrocitronellol, and the like.

In one embodiment, the monomer is commercially available. In one embodiment, the monomer is selected from the group consisting of pentaerythriol triacrylate (PETIA), trimethylolpropane triacrylate (TMPTA), bisphenol-A epoxy diacrylate (Ebecryl 605), tripropylene glycol diacrylate (TPGDA), and bisphenol-A diglycyl ether (BADGE).

In one embodiment, the initiator comprises at least one functionality that is readily deprotonated by a base catalyst. In one embodiment, the initiator comprises at least one thiol functionality that is readily deprotonated by a base catalyst. In one embodiment, the initiator comprises at least two thiol functionalities that are readily deprotonated by a base catalyst. Exemplary initiators include, but are not limited to, trimethylolpropane tris(3-mercaptopropionate) (TT1), Capcure 3-80, trimethylolpropane tris (3-mercaptoacetate), glycol di-3-mercaptopropionate, polypropylene glycol bis (3-mercaptopropionate), ethoxylated trimethylolpropane tris (3-mercaptopropionate), glycol dimercaptoacetate, ethoxylated glycol dimercaptoacetate, 1, 4-bis(3-mercaptobutyryloxy) butane, tris [2-(3-mercaptopropionyloxy) ethyl]isocyanurate, 1,3,5-tris (3-mercaptobutoxyethyl-1,3,5-triazine-2,4,6(1H,3H,5H)-trione, pentaerythritol tetrakis(3-mercaptopropionate), pentaerythritol tetrakis(2-mercaptoacetate), 1,6-hexanedithiol, 1,3-propanedithiol, 1,2-ethanedithiol, polyethylene glycol dithiol comprising 1-10 ethylene glycol repeating units, or combinations thereof.

In one embodiment, the molar ratio of monomer to initiator is about 1:1. In one embodiment, the molar ratio of monomer to initiator is about 1.1:1. In one embodiment, the molar ratio of monomer to initiator is about 1.2:1. In one embodiment, the molar ratio of monomer to initiator is about 1.3:1. In one embodiment, the molar ratio of monomer to initiator is about 1.4:1. In one embodiment, the molar ratio of monomer to initiator is about 1.5:1. In one embodiment, the molar ratio of monomer to initiator is about 1.6:1. In one embodiment, the molar ratio of monomer to initiator is about 1.7:1. In one embodiment, the molar ratio of monomer to initiator is about 1.8:1. In one embodiment, the molar ratio of monomer to initiator is about 1.9:1. In one embodiment, the molar ratio of monomer to initiator is about 2:1.

In one embodiment, the silica comprises fumed silicas, precipitated silicas, silica sols, silica gels, pyrogenic silicas, silica compounds of natural or synthetic origin, aluminosilicates, smectites, magnesium silicates, clays, wollastonite, talc, mica, attapulgite, sepiolite, montmorillonite and/or bentonites.

In one embodiment, the silica comprises a hydrophilic silica. In one embodiment, the silica comprises a hydrophobically modified silica. In one embodiment, the surface of the silica is modified with at least one organic component, such as surface-modified silicas. Surface modification is understood to mean the chemical and/or physical attachment of organic components to the surface of the silica particles. In other words, in the case of surface-modified silicas, at least part of the surface of at least some of the silica particles is covered with the surface modifiers. In the present case, the silicas are silanized by reacting fumed silica with trimethylchlorosilane or trimethylsilanol or hexamethyldisilazane in a known manner, the trimethylsilyl groups being fixed on the surface of the silica.

In one embodiment, the silica has a specific surface area of at least about 100 m²/g. In one embodiment, the silica has a specific surface area of about 100 to about 300 m²/g. In one embodiment, the silica has a specific surface area of about 150 to about 250 m²/g. In one embodiment, the silica has a specific surface area of about 175 to about 225 m²/g.

In one embodiment, the amount of silica in the composition is about 0 to about 10 phr or about 0.1 to about 10 phr. In one embodiment, the amount of silica in the composition is about 1 to about 10 phr. In one embodiment, the amount of silica is about 2 to about 9 phr. In one embodiment, the amount of initiator is about 3 to about 8 phr. In one embodiment, the amount of initiator is about 4 to about 7 phr. In one embodiment, the amount of initiator is about 5 to about 6 phr.

In one embodiment, the milled carbon fiber comprises a commercial or synthetic milled carbon fiber. In one embodiment, the milled carbon fiber is manufactured from polyacrylonitrile (PAN) precursor. In one embodiment, the milled carbon fiber comprises a commercial carbon fiber such as Zoltek®PX35. In one embodiment, milled carbon fiber comprises a high purity carbon fiber such as Zoltek®PX30. In one embodiment, milled carbon fiber comprises an oxidized PAN fiber such as Zoltek®OX. Though one manufacturer is described herein, any manufacturer of milled carbon fiber is similarly considered herein.

In one embodiment, the loading of milled carbon fiber is about 5 to about 50 phr. In one embodiment, the amount of milled carbon fiber is about 10 to about 40 phr. In one embodiment, the amount of milled carbon fiber is about 15 to about 30 phr. In one embodiment, the amount of milled carbon fiber is about 15 to about 25 phr.

In one embodiment, the resin composition further comprises a non-skid component. In one embodiment, the non-skid component minimizes the slipperiness of any resulting polymer material formed form polymerization of the resin composition. In one embodiment, the non-skid material increases the coefficient of friction of a coating comprising the resin composition. In one embodiment, the non-skid material is a granular/particulate material. Exemplary non-skid materials include, but are not limited to crushed/milled glass, silica, aluminum oxide, and the like. In one embodiment, the resin composition comprises aluminum oxide. In one embodiment, the non-skid material is 14 mesh/16 grit. In one embodiment, the non-skid material is 30 mesh/36 grit. In one embodiment, the non-skid material is 46 mesh/54 grit. In one embodiment, the loading of non-skid component is about 0 to about 200 phr. In one embodiment, the loading is about 10 to about 100 phr. In one embodiment, the loading is about 25 phr to about 100 phr.

In one embodiment, the resin composition further comprises a filler. Examples of fillers useful herein include, but are not limited to, hollow or solid ceramic microspheres, clays, aluminum iron magnesium silicate, aluminum silicon oxide, aluminum silicate, calcium magnesium carbonate, calcium silicate hydrate, calcium carbonate, calcium metasilicate, silica anhydrite+kaolinite, magnesium aluminum silicate hydrate, magnesium aluminum silicate hydrate, magnesium silicate, magnesium silicate hydrate, silicon dioxide, silicon oxide, and mixtures thereof. The above list of compounds is not meant to be limiting and various other fillers known in the art are also considered and should be considered to be within the scope of the present disclosure. In a further aspect, the average particle size of the filler is between 270 mesh (0.053 mm) and 400 mesh (0.037 mm) or is about 325 mesh (0.044 mm). In another aspect, the average particle size of the filler is 270 mesh, 300 mesh, 325 mesh, 350 mesh, 375 mesh, or 400 mesh, where any value can be a lower or upper endpoint of a range. In another aspect, the particle size of the filler is from 1 μm to 10 μm (90^{th} percentile) or is 1, 2, 3, 4, 5, 6, 7, 8, 9, or 10 μm, or is about 7.7 μm, where any value can be a lower or upper endpoint of a range.

In one aspect, the filler loading is from about 1 to about 30 phr based on the total of reactive monomer. In another aspect, the amount of filler can be about 1, 5, 10, 15, 20, 25, or 30 phr. In any of these aspects, the filler can contribute to overall mechanical stability and strength of the anti-skid composition and can assist in reducing the drying time of the anti-skid composition. In a further aspect, the filler imparts increased chemical resistance to the anti-skid composition.

In one embodiment, the resin composition further comprises a thickener. Various thickeners are contemplated. Examples of thickeners useful herein include, but are not limited to, cellulose and related polymers such as, for example, hydroxyethyl cellulose, hydroxypropyl cellulose, hydroxyethyl methyl cellulose, hydroxypropyl methyl cellulose, ethyl hydroxyethyl cellulose, carboxymethyl cellulose, and combinations thereof.

In one embodiment, the thickener can be from about 0.01 to 1 phr, or can be about 0.1, about 0.2, about 0.3, about 0.4, about 0.5, about 0.6, about 0.7, about 0.8, about 0.9, or about 1 phr. In one aspect, the thickener keeps the filler in suspension and/or prevents the filler from settling. In this aspect, preventing the filler from settling leads to a longer shelf life for the finished product.

In one embodiment, the resin composition further comprises plasticizer. Various phthalate and non-phthalate plasticizers are contemplated. In one aspect, the plasticizer is a non-phthalate plasticizer. In another aspect, the plasticizer sold under the trade names EASTMAN 168® (i.e., 1,4-benzenedicarboxylic acid, bis(2-ethylhexyl) ester) or BENZOFLEX® 2088 benzoate plasticizer manufactured by Eastman Chemical Company is useful as the plasticizer. In another aspect, K-FLEX® 500 dibenzoate plasticizer manufactured by Kalema is useful as the plasticizer. In one aspect, the plasticizer lowers the overall glass transition temperature of the composition without softening and/or weakening the mechanical stability of the composition.

In one aspect, the present disclosure relates, in part, to a polymerized coating disposed over a substrate, the coating comprising: a repeat unit comprising a monomer comprising at least one reactive functionality, wherein the reactive functionality is an alkenyl group or a heterocyclic group; a crosslinker formed from an initiator comprising at least one thiol functionality; wherein the polymerized coating further comprises: about 0-10 or about 0.1-10 phr silica; about 5-50 phr milled carbon fiber; about 0-5 or about 0.1-5 phr of at least one source of urease; about 0-5 or about 0.1-5 phr phenyl phosphonic acid; and about 0-5 or about 0.1-5 phr substituted or unsubstituted phenol.

In one embodiment, the monomer and initiator are as described herein.

In one embodiment, the coating comprises a copolymer. In one embodiment, the coating comprises at least two monomers with different reactive functionalities.

In one embodiment, the coating further comprises a non-skid component as described herein. In one embodiment, the coating further comprises a filler component.

In one embodiment, the coating is disposed over a substrate as described herein.

In one embodiment, an additional layer is disposed between the substrate and the coating of the present disclosure. In one embodiment, the additional layer comprises a primer which enhances the binding of the coating to the substrate. In one embodiment, the additional layer comprises an epoxy resin. There is no particular limit to the composition of the additional layer, and any primer material known in the art may be used.

In one embodiment, the coating has a pot life of about 1 day to about 90 days. In one embodiment, the coating has a pot life of about 1 day to about 80 days. In one embodiment, the coating has a pot life of about 1 day to about 70 days. In one embodiment, the coating has a pot life of about 10 days to about 90 days. In one embodiment, the coating has a pot life of about 20 days to about 90 days. In one embodiment, the coating has a pot life of about 30 days to about 90 days. In one embodiment, the coating has a pot life of about 40 days to about 90 days. In one embodiment, the coating has a pot life of about 50 days to about 90 days. In one embodiment, the coating has a pot life of about 60 days to about 90 days. In one embodiment, the coating has a pot life of about 70 days to about 90 days. In one embodiment, the coating has a pot life of about 80 days to about 90 days.

In one embodiment, the coating is a non-skid coating. In one embodiment, the coating has a coefficient of friction (COF) value of greater than 1.4 when the coating is dry. In one embodiment, the dry coating has a COF of greater than 1.5. In one embodiment, the dry coating has a COF of greater than 1.6. In one embodiment, the dry coating has a COF of greater than 1.7. In one embodiment, the dry coating has a COF of greater than 1.8. In one embodiment, the dry coating has a COF great than or equal to 1.8.

In one embodiment, the coating has a coefficient of friction (COF) value of greater than 1.1 when the coating is wet. In one embodiment, the wet coating has a COF of greater than 1.2. In one embodiment, the wet coating has a COF of greater than 1.3. In one embodiment, the wet coating has a COF of greater than 1.4. In one embodiment, the wet coating has a COF of greater than 1.5. In one embodiment, the dry coating has a COF great than or equal to 1.5.

In one embodiment, the coating is resistant to impact. In one embodiment, the coating shows little or no wear upon repeated impact with a 4 lb weight. In one embodiment, the coating has an impact strength of about 1 kJ/m² to about 10 kJ/m². In one embodiment, the coating has an impact strength of about 2 kJ/m² to about 10 kJ/m². In one embodiment, the coating has an impact strength of about 3 kJ/m² to about 10 kJ/m². In one embodiment, the coating has an impact strength of about 4 kJ/m² to about 10 kJ/m². In one embodiment, the coating has an impact strength of about 5 kJ/m² to about 10 kJ/m². In one embodiment, the coating has an impact strength of about 6 kJ/m² to about 10 kJ/m². In one embodiment, the coating has an impact strength of about 7 kJ/m² to about 10 kJ/m². In one embodiment, the coating has an impact strength of about 8 kJ/m² to about 10 kJ/m². In one embodiment, the coating has an impact strength of about 9 kJ/m² to about 10 kJ/m².

In one embodiment, the coating shows no delamination or softening when submerged in any or all of ethanol, salt water, detergent, anti-freeze, or motor oil. In one embodiment, the coating prevents oxidation of the substrate. In one embodiment, the coating prevents the substrate from rust. In one embodiment, the coating serves as an adhesive.

Now having described various embodiments of the present disclosure, additional feature are provided.

Feature 1: A method of depositing a coating over a substrate, the method comprising the steps of: providing a resin composition; applying the resin composition over a substrate; exposing the resin composition to a solution comprising a base catalyst; and polymerizing the resin composition; wherein the resin composition comprises: a monomer comprising at least one reactive functionality, wherein the reactive functionality is an alkenyl group or a heterocyclic group; an initiator comprising at least one thiol functionality, wherein the initiator is capable of being activated using a base catalyst; wherein the resin composition further comprises: about 0.1-10 phr silica; about 5-50 phr milled carbon fiber; and about 0.1-5 phr of at least one source of urease. Feature 2. The method of the features described above and herein, wherein the solution comprising a base catalyst comprises at least one compound selected from the group consisting of: ammonia, urea, or substituted or unsubstituted: amine, hydroxylamine, and combinations thereof. Feature 3. The method of the features described above and herein, wherein the at least one source of urease is selected from the group consisting of: watermelon seeds, jack beans, pea seeds, soybeans, sword beans, ruminal urease, and synthetic urease. Feature 4. The method of the features described above and herein, wherein the solution comprising a base catalyst is about 5% to about 30% base. Feature 5. The method of the features described above and herein, wherein the resin composition is exposed to the solution comprising a base catalyst for about 5 minutes to about 60 hours. Feature 6. The method of the features described above and herein, wherein the solution comprising a base catalyst comprises urea. Feature 7. The method of the features described above and herein, wherein the solution comprising a base catalyst is about 5% to about 20% urea. Feature 8. The method of the features described above and herein, wherein the solution comprising a base catalyst is further acidified.

Feature 9. A resin composition comprising: a monomer comprising at least one reactive functionality, wherein the reactive functionality is an alkenyl group or a heterocyclic group; an initiator comprising at least one thiol functionality, wherein the initiator is capable of being activated using a base catalyst; wherein the resin composition further comprises: about 0.1-10 phr silica; about 5-50 phr milled carbon fiber; and about 0.1-5 phr of at least one source of urease. Feature 10. The composition of the features described above and herein, wherein the monomer further comprises a moiety selected from the group consisting of: bisphenol, glycol, alcohol, quaternary carbon, derivatives thereof, and combinations thereof. Feature 11. The composition of the features described above and herein, wherein the reactive functionality is selected from the group consisting of: a methacrylate group, a malonate group, a maleimide group, a vinyl group, an acrylate, an epoxide, an oxetane, and an aziridine. Feature 12. The composition of the features described above and herein, wherein the monomer is selected from the group consisting of: an acrylate, a diacrylate, a triacrylate, an epoxy acrylate, an epoxy diacrylate, an epoxy triacrylate, a glycidyl ether, a diglycidyl ether, and a triglycidyl ether, and combinations thereof. Feature 13. The composition of claim 9, wherein the initiator comprises at least two thiol functionalities. Feature 14. The composition of the features described above and herein, wherein the initiator comprises a compound selected from the group consisting of trimethylolpropane tris(3-mercaptopropionate) (TT1), trimethylolpropane tris (3-mercaptoacetate), glycol di-3-mercaptopropionate, polypropylene glycol bis (3-mercaptopropionate), ethoxylated trimethylolpropane tris (3-mercaptopropionate), glycol dimercaptoacetate, ethoxylated glycol dimercaptoacetate, 1, 4-bis(3-mercaptobutyryloxy) butane, tris [2-(3-mercaptopropionyloxy) ethyl]isocyanurate, 1,3,5-tris (3-mercaptobutoxyethyl-1,3,5-triazine-2,4,6(1H,3H,5H)-trione, pentaerythritol tetrakis(3-mercaptopropionate), pentaerythritol tetrakis(2-mercaptoacetate), 1,6-hexanedithiol, 1,3-propanedithiol, 1,2-ethanedithiol, polyethylene glycol dithiol comprising 1-10 ethylene glycol repeating units, or combinations thereof. Feature 15. The composition of the features described above and herein, wherein the molar ratio of monomer to initiator is about 1:1. Feature 16. The composition of the features described above and herein, wherein the silica comprises fumed silica, precipitated silica, silica sol, silica gel, or pyrogenic silica. Feature 17. The composition of the features described above and herein, wherein the at least one source of urease is selected from the group consisting of watermelon seeds, jack beans, pea seeds, soybeans, sword beans, ruminal urease, and synthetic urease. Feature 18. The composition of the features described above and herein, wherein the solution comprising a base catalyst comprises urea.

Feature 19. A polymerized coating disposed over a substrate, the coating comprising: a repeat unit formed from a monomer comprising at least one reactive functionality, wherein the reactive functionality is an alkenyl group or a heterocyclic group; a cross-linker formed from an initiator comprising at least one thiol functionality; wherein the polymerized coating further comprises: about 0.1-10 phr silica; about 5-50 phr milled carbon fiber; about 0.1-5 phr of at least one source of urease; about 0.1-5 phr phenyl phosphonic acid; and about 0.1-5 phr substituted or unsubstituted phenol.

### EXAMPLES

The present disclosure is further described in detail by reference to the following experimental examples. These examples are provided for purposes of illustration only, and are not intended to be limiting unless otherwise specified. Thus, the present disclosure should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present disclosure and practice the claimed methods. The following working examples therefore are not to be construed as limiting in any way the remainder of the disclosure.

The present disclosure is drawn to, in part, a novel approach to cure-on-demand thiol-acrylate and thiol-epoxy coatings using ammonia (NH₃) as the catalyst. A mixture of thiol and acrylate or a thiol and epoxy are applied to a surface which can be cured in two ways: 1) Ammonia solution (40%) can be applied. The ammonia gas diffuses from the solution into the resin mixture and catalyzes the reaction of the thiol with the acrylate or epoxy. Aluminum foil or aluminized mylar sheets are placed over the coating to prevent evaporation of the ammonia. 2) Curing can be initiated by using the in situ generation of ammonia by the urease-catalyzed hydrolysis of an aqueous solution of urea. Watermelon seed powder (WMSP) is mixed into the resins. The urease present in the WMSP catalyzes the aqueous urea to produce ammonia that then diffuses into the resin. The curing is activated by spraying an acidic aqueous solution of urea over the resin mixture applied to the surface. The system is covered with aluminum foil or aluminized mylar sheets to optimize the ammonia production into the system and to prevent ammonia from evaporation. The urease from watermelon seeds catalyzes the production of ammonia from an acidic solution of urea with a time delay dependent on the initial pH, which allows covering the layer before ammonia is produced; the amount of ammonia lost to the atmosphere is thus reduced (Mai et al., 2021, Communications Chemistry, 4, 1). The curing of these coatings proceeds by the base-catalyzed Michael addition of a multifunctional thiol to a multifunctional acrylate or multifunctional epoxy. As the base-ammonia gas diffuses into the layer, it deprotonates the thiol, allowing addition to the acrylate or epoxy (Hoyle et al., 2010, Chemical Society Reviews, 39, 1355). The thiol-acrylate systems cured in less than 24 hours while the epoxy systems required more time. The fillers were added to increase the mechanical strength of coatings. The present method provides the following advantages: a one-pot formulation that requires no mixing; 100% solids with zero volatile organic compounds (VOCs); after the resin has been applied, it does not react until a layer of ammonia solution or urea is applied; and the beginning of the reaction can be controlled by adjusting the pH of the urea solution.

Thiol chemistry has gained significant interest in the last ten years due to its effectiveness and adaptability, with a variety of thiol reactions—the most well-known of which is the click reaction. Thiol chemistry can be split into two groups: radical-mediated reactions connected to the thiol-ene and thiol-yne reactions and base-catalyzed nucleophilic reactions associated with the thiol-epoxy, thiol-isocyanate, and thiol Michael addition reactions (Dondoni, 2008, Angewandte Chemie International Edition, 47, 8995; Hoyle, 2010 Angewandte Chemie International Edition, 49, 1540; Hoyle et al., 2010, Chemical Society Reviews, 39, 1355; Lowe et al., 2010, Journal of Materials Chemistry, 20, 4745; Hoyle et al., 2004, Journal of Polymer Science Part A: Polymer Chemistry, 42, 5301; Kade et al., 2010, Journal of Polymer Science Part A; Polymer Chemistry, 48, 743; Fu, 2011, Polymer Chemistry, 2, 465; Lowe, 2010, Polymer Chemistry, 1, 17). Among these, Michael addition reaction has been investigated for coatings applications since the 1980s. A Michael addition, in general, is the addition of a nucleophile to an electron deficient carbon-carbon double bond and was named after Arthur Michael, who first reported them in 1887 (Michael, 1887, Am Chem J, 9, 115). The first Michael addition reactions were carried out by Arthur Michael in the late 1880s to explain the cyclopropane derivative synthesis seen by Conrad and Kuthzeit in a reaction between diethyl 2,3-dibromopropionate and diethyl sodiomalonate (Tokoroyama, “Discovery of the Michael Reaction”, Wiley Online Library, 2010). Due to the benefits of thiol-click chemistry, nucleophilic thiol reactions, such as base-catalyzed thiol-epoxy, thiol-isocyanate, and thiol-acrylate Michael addition reactions, have been successfully employed to quickly and efficiently create cross-linked polymers (high conversion, solvent free, oxygen-resistant, etc.) (Jin et al., 2015, Polymer, 81, 70; Nair et al., 2014, Chemistry of Materials, 26, 724). Michael addition reactions have traditionally been widely utilized in organic synthesis; their application in materials applications, surface modification, polymer modification, and polymer synthesis generally developed concurrently with the wider adoption of click chemistry in materials. The flexibility of the weak sulfur-hydrogen bond makes it possible to start the thiol-epoxy and thiol-Michael addition reaction using a variety of precursor components. The presence of the highly reactive sulfur within the thiol functional group, also known as mercury-scavenging mercaptans historically, has ensured its use in applications that call for enhanced physical and chemical properties. For instance, sulfur has been used to cross-link polymers since the beginning of the Industrial Revolution (Mather et al., 2006, Progress in Polymer Science, 31, 487; Chan et al., 2009, Journal of the American Chemical Society, 131, 5751). The Michael reaction has also been used to make linear, graft, hyperbranched, dendritic, and network polymers (Tokura et al., 1983, Polymer Journal, 15, 553). It lends itself to both step growth and chain growth polymerization (Beylen et al., 1988, Polysiloxane Copolymers/Anionic Polymerization, 87). There are two kinds of this step-growth polymerization: polycondensation and polyaddition reactions. The polyaddition-step growth reaction method yields no byproducts (Zhang et al., 2012, Polymer Science: A Comprehensive Reference, 10 Volume Set, 2012, 7).

The present disclosure is further drawn to, in part, a one-pot coating formulation of the thiol-epoxy click reaction and the thiol-acrylate Michael addition reaction, both of which were driven to occur via a step-growth polymerization-polyaddition mechanism. Both the thiol-epoxy and thiol-acrylate coatings were cured by an ammonia-catalyzed addition reaction. Initial studies on curing mechanism required a 28-30% aqueous solution of ammonia, which can present health risks due to its high diffusivity (Sommer, 2013, Animal Manure Recycling. John Wiley & Sons, Ltd, 2013, 131). Following these concerns, a comparatively safer technique was developed using a natural source of ammonia production within the polymerizing media.

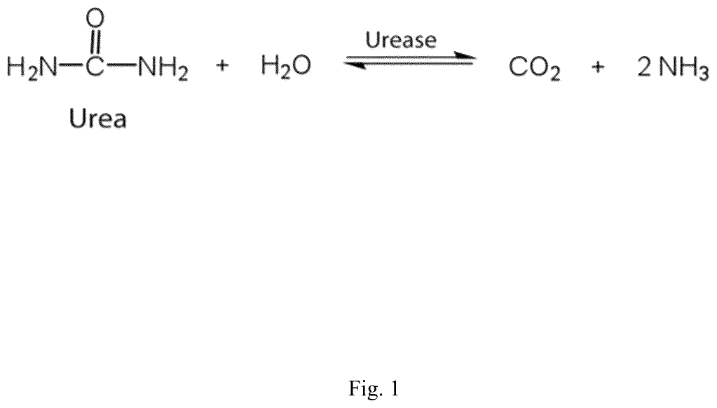

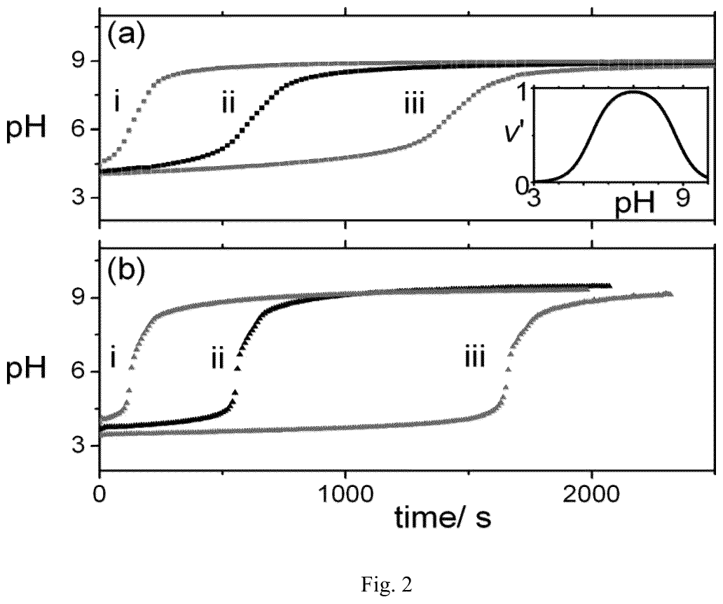

The present disclosure uses pH clock reactions to trigger polymerization and gelation. Such clock reactions exhibit a large pH change after a programmable “clock time”. The first system was a formaldehyde clock reaction to trigger the thiol-acrylate Michael addition (Hu et al., 2010, J. Polym. Sci. Part A: Polym. Chem., 48, 2955). To avoid the toxicity of formaldehyde, a urea-urease system was utilized (FIG. 1). The hydrolysis of urea to produce ammonia and carbon dioxide is catalyzed by urease. Hu et al. showed that the production of base and the pH dependence of the urea-urease reaction gives rise to base-catalyzed feedback (Hu et al., 2010, J. Phys. Chem. B, 114, 14059) (FIG. 2).

Jee et al. showed that the urea-urease reaction could be used to create a time-lapse polymerization of a thiol-acrylate solution (Jee et al., 2016, Angew. Chem., 128, 2167). Mai et al. demonstrated that an extract from watermelon seeds provides a stable form of urease (Mai et al., 2021, Communications Chemistry, 4, 101). This watermelon seed powder contains urease that is stable to at least a year at ambient conditions.

Ammonia can serve as a catalyst for the Michael addition of a thiol to an acrylate and for the reaction of a thiol with an epoxy. A 30% solution of aqueous ammonia contains a small fraction of NH₄OH; almost all the nitrogen is present as NH₃ (Kissel, “Encyclopedia of Soils in the Environment”, 2005, 56). Directly applying aqueous ammonia solution to a solution of a thiol and acrylate or a thiol and epoxy will result in a curing of the layer as the ammonia diffuses into the layer.

To avoid the potential hazards of ammonia vapors from applying a 30% solution of aqueous ammonia onto a layer of reactants, the application of an acidic solution of urea onto the resin formulation containing WMSP was explored. The conversion of urea to ammonia by urease was delayed by the low pH, and so a time delay was created to allow the coating to be covered with aluminum foil before the ammonia was produced.

The experimental details will now be described herein.

### Materials and Methods

Trimethylolpropane triacrylate (TMPTA), Ebecryl®605 and pentaerythritol triacrylate (PETIA) were purchased from Allnex (Alpharetta, GA). Trimethylolpropane tris(3-mercaptopropionate) (TT1), Phenylphosphonic acid (PPA), and Bisphenol a diglycidyl ether (BADGE) were purchased from TCI America. and Trimethylolpropane triglycidyl ether (TMPTE) was purchased from Sigma-Aldrich. 4-methoxyphenol (MeHQ) were obtained from Sigma Aldrich.

Fumed silica (FS) (Aerosil®200, 175-225 m2/g BET surface area) was obtained from Evonik Industries (Parsippany, NJ). Zoltek®PX35 (referred to as Zoltek or milled carbon fiber (CF), 150×7.2 squared microns) was provided by Zoltek Companies, Inc. (St. Louis, MO). Aluminum oxide (16 grit, 14 mesh, 1.2 mm) was obtained from Floorguard Products, Inc.

### Preparation of Coatings Formulation

The Thiol-Epoxy coating formulation was fabricated following 1:1 stoichiometric ratio of thiol to epoxy compounds and the same texture was maintained for the preparation of thiol-acrylate coatings. These formulations were facing the critical challenge of very short pot lives. Particularly most of the thiol-acrylate mixtures were reacting within an hour and the thiol-epoxy was having relatively longer pot lives than acrylates. All additional components were added in parts per hundred resin (phr), which is the amount of material added (in grams) for every 100 grams of resin. To stabilize the pot lives of these formulations, 1.0 phr 4-methoxy phenol was added to inhibit carbon centered radical polymerization of acrylates and 0.50 phr phenyl phosphonic acid (PPA) was added to suppress the pH of thiols from increase. To increase viscosity and mobility of mixtures, 4 phr of fumed silica was added to the formulation. 50 phr of aluminum oxide was added to the formulation followed by 20 phr of milled carbon fiber. Both the fillers contributed to increasing the mechanical strength of cured coatings.

Different sized steel panels coated with an epoxy primer (Interbond®998, from International Paint) were used as substrates. The panels were previously primed, and all coatings in this work were applied after the overcoat window.

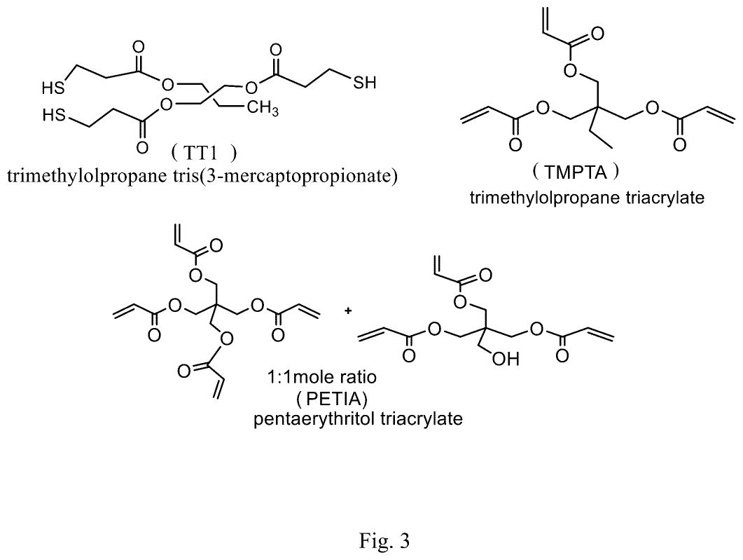

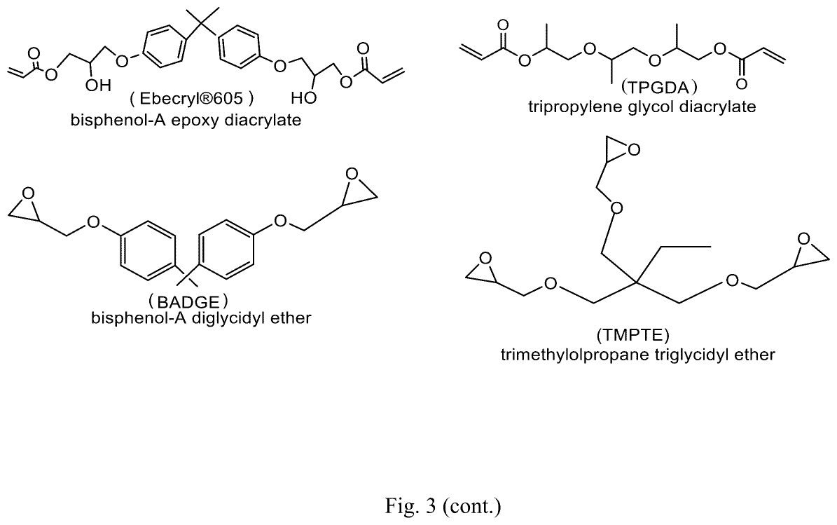

Alternatively, following the correct stoichiometric ratio of thiol to monomer (structures shown in FIG. 3), resins such as 1:1 ratio of thiol to epoxy and 1:1 ratio of thiol to acrylate were mixed in a pot respectively. Fillers such as 22.0 phr of milled carbon fiber and 2.5 phr of fumed silica were added to every formulation. 0.25 phr of phenyl phosphonic acid and 0.5 phr. of 4-methoxy phenol were added to extend the pot life.

To produce ammonia gas in situ, urease enzyme from watermelon seed powder (WMSP) was used, and a 7.5% prepared aqueous urea solution was sprayed evenly over the whole coatings applied on the steel panel. 0.5 phr of WMSP was previously mixed with the resins.

### Curing Strategy

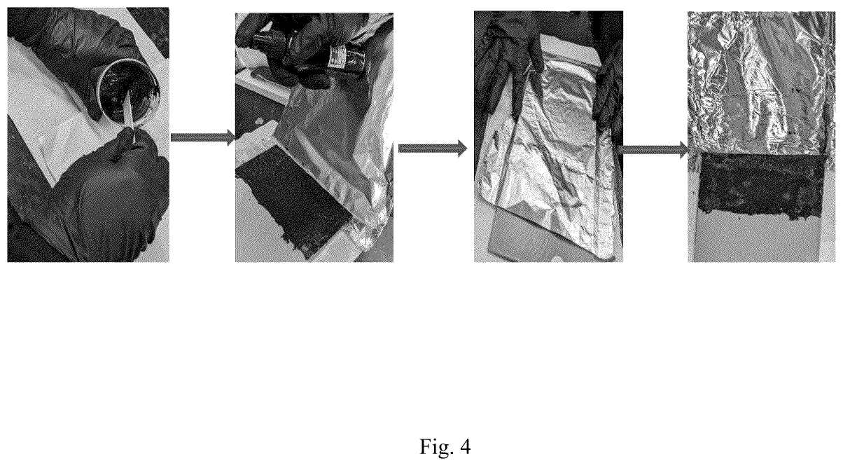

Watermelon seed powder (WMSP) can be used in two ways to produce ammonia gas in situ: either by mixing it with the resins or by dispersing it on top of coatings after they have been applied to the substrate. Around 0.5-1 phr of 10 micron size watermelon seed powder (WMSP) was used as a source of urease enzyme, and a 12% aqueous acidic urea solution was sprayed on top of the coatings on the substrate ([NH₃]=4.54 M). Thus, the curing of coatings was activated by a urea-urease reaction or spraying a commercially available 28-30% aqueous solution of ammonia ([NH₃]=14.8 M). The loss of ammonia was prevented by covering the coatings with aluminum foil (FIG. 4). As the ammonia gas diffuses into the layer, it deprotonates the thiol, allowing addition to the acrylate or epoxy part of the resins.

Curing reactions were set to room temperature. However, some samples were examined at around 5° C. and some were at around 40° C. Thickness of the coatings were around 1 mm. In both processes, coatings on the metal panel were kept covered with aluminum foil until curing to prevent the ammonia gas from evaporating. To check the status of coating curing, the top of aluminum foil was pressed with a finger to test if the coating was hard. Aluminum foil is opened to conduct an actual curing test once the coating has cured and feels hard to the touch. Further, curing was also confirmed by the disappearance of monomer's functional groups using IR spectral analysis.

Alternatively, approximately 30% ammonia containing ammonium hydroxide solution was sprayed to cure the thiol-epoxy/thiol-acrylate coatings on steel panels coated with an epoxy primer (Interbond®998, from International Paint) were used as the substrates. Later, safer production of ammonia into the curing system was developed. Watermelon seed powder (WMSP) and urea solution were used to produce ammonia. The water which was used to prepare urea solution was made acidic to delay the ammonia gas production. The presence of the urease enzyme in WMSP causes nucleophilic base-ammonia gas to be generated when urea aqueous solution interacts with it. In both processes, coatings on the metal panel were kept covered with aluminum foil until curing to prevent the ammonia gas from evaporating.

### Calculation of Monomer Conversion Percentage by FTIR Data

The conversion of thiol, epoxy, and acrylate groups was monitored using a Bruker Tensor 27 Fourier transform infrared (FTIR) spectrometer fitted with a Pike Miracle single-bounce diamond attenuated total reflectance (ATR) cell. The data was collected between 400 and 4000 cm-1, with a resolution of 4 cm-1. Sixteen scans were done for each spectrum, and all samples were solid polymer. Opus, Bruker's FTIR software program, was used to process the FTIR data after it was obtained. The Thiol group concentration was tracked using the S—H stretching peak, which has a center point of 2573 cm-1, and acrylate group concentration was found using the C═C stretching band, which has a center point of 1625 cm-1.

$$Percentage Conversion = \frac{{M}_{m} - {M}_{p}}{{M}_{m}} \times 100$$

Mₘ is the monomer peak area and Mₚ is the peak area after polymerization.

### Mechanical Performance Test

Scratch, scrape, and impact testing were used to evaluate the mechanical performance of the coatings. Impact testing was performed in accordance with ASTM G14 guidelines. A 4-pound weight was dropped onto an indenter. Each impact test involved 25 impacts in a 3″×3″ area. Next, any coating that had become loose around the impact zone was removed with a 1-inch chisel. The number of links among adjacent effects was counted. The score was calculated by multiplying the sum of the connections by 2.5 and subtracting from 100. The percentage of undamaged coating that is still present between the impact locations is reflected in the overall score.

### Chemical Resistance Test

The relative resistance of coatings against a variety of chemicals, such as ethanol, natural saltwater, motor oil, detergent, and deicing defrosting fluid, was ascertained through qualitative chemical testing. Each coating was placed in a 1,400 mL beaker and halfway submerged in ethanol for 24 hours, deicing/defrosting fluid for 24 hours, then seawater, motor oil, or detergent for 4 weeks. The beakers were then sealed with aluminum foil. Following removal, a 1-inch chisel was used to check for coating softening or loss of adherence on each coating substrate. Together with the impacted versus non-impacted coating, the unsubmerged and immersed portions were compared. Prior to examination, the coatings immersed in ethanol and deicing/frosting solution were allowed to recover for six hours.

The results and discussion of the experiments performed will now be discussed herein.

### Results and Discussion

The thiol-epoxy system was polymerized and deposited on the substrate by ammonia nucleophile catalyzed reaction. Thiolate anion, which is produced when an acidic thiol group is deprotonated by an ammonia catalyst, attacks the electron-deficient epoxy ring, causing a ring opening polymerization to occur. For thiol-acrylate system, thiols are also deprotonated by the ammonia catalyst and added with electron deficient acrylate carbon through a conjugate addition/Michael addition reaction mechanism. Epoxy resins have several benefits, including improved physical and mechanical characteristics, little shrinkage during polymerization, and stoichiometric interactions between the epoxy functional groups and thiols (Jian et al., 2013, Journal of Materials Chemistry C, 1, 4481; Sangermano et al., 2010, Polymer International, 59, 1046; Nair et al., 2012, Polymer, 53, 2429). In both the synthetic and natural ammonia curing techniques, thiol-acrylates were observed in this experiment to cure faster than thiol-epoxy. Acrylates can extract a hydrogen from the thiol monomers (step-growth mechanism) and easily homopolymerize via a chain-growth mechanism (Matsushima et al., 2010, J. Polym. Sci. A: Polym. Chem., 48, 3255; Chan et al., 2010, Macromolecules, 43, 6381). There are two probable reactions in which the radical can take part in a mixture of acrylate and thiol monomers: either adding to another acrylate functional group or chain-transfer steps to the thiol (Chan et al., 2009, European Polymer Journal, 45, 2717). The coating's overall performance was also found to be dependent on adding fillers. Coatings were cured without fillers but were mechanically weak and fillers also helped rapid curing of coatings. Fumed silica and carbon fiber helped increase the viscosity and tensile strength of the formulations and cured coatings respectively.

### Thiol-Acrylate Conversion

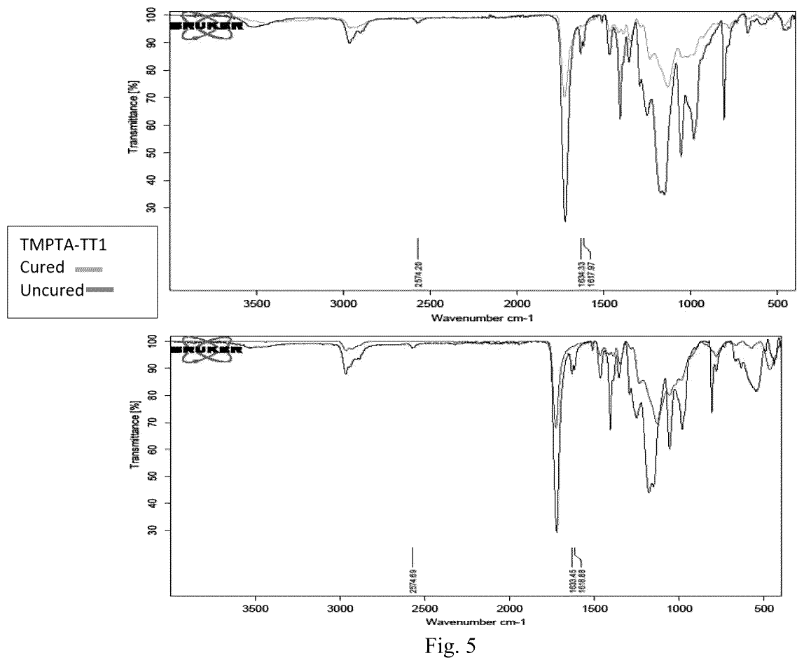

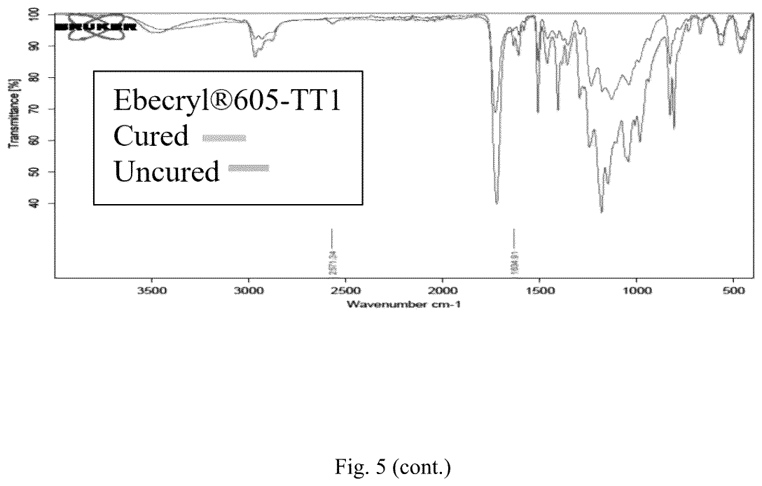

The thiol's S—H stretching frequency of around 2575 cm⁻¹ and acrylate group's C═C stretching frequency of around 1633 cm⁻¹ were detected in ATR. After curing, both the thiol and acrylate peaks disappeared, these functional group's conversions confirmed that a polymerization reaction took place (FIG. 5).

### Thiol-Epoxy Conversion

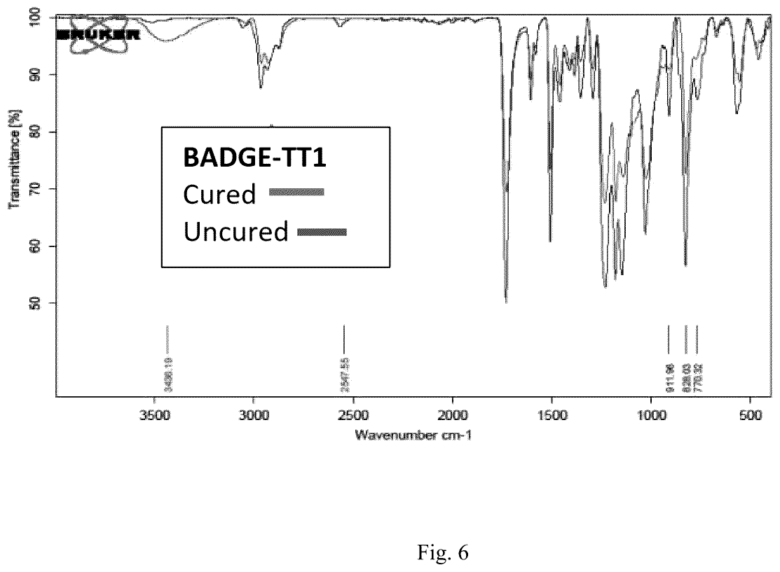

Before curing of thiol-epoxy coatings, thiol's S—H stretching frequency of ˜2548-2580 cm⁻¹ was detected in ATR. After curing, the thiol's band disappeared, and the newly formed O—H bond's stretching frequencies appeared as a broad peak at 3438 cm⁻¹. The peaks for epoxied C—O's are around 800-930 cm⁻¹ whose intensity decreased significantly after polymerization These functional group's conversions confirmed that a polymerization reaction took place (FIG. 6).

TABLE 1
Monomer Conversion Percentage

| Formulations containing | Monomer's Functional |
|---|---|
| FS, CF, & Al₂O₃ | Conversion % |
| TMPTA-TT1 | Thiol, —SH: 100 & Acrylate, |
| | C═C: 99.64 |
| PETIA-TT1 | Thiol, —SH: 100 & Acrylate, |
| | C═C: 98.28 |
| Ebecryl ®605-TT1 | Thiol, —SH: 100 & Acrylate, |
| | C═C: 100 |
| BADGE-TT1 | Thiol, —SH: 92.06 & Epoxy, |
| | C—O: 90.90 |
| TMPTE-TT1 | Thiol, —SH: 91.18 & Epoxy, |
| | C—O: 98.39 |

From the above IR peak analysis, it is clear that most of the thiol (—SH), epoxy (C—O), and acrylate (C═C) bonds almost completely disappeared after polymerization of the monomers. Expectedly, the functional conversion percentage Table 1 testifies that the higher conversion of those functional groups was achieved.

### Curing Status of Coating Formulations

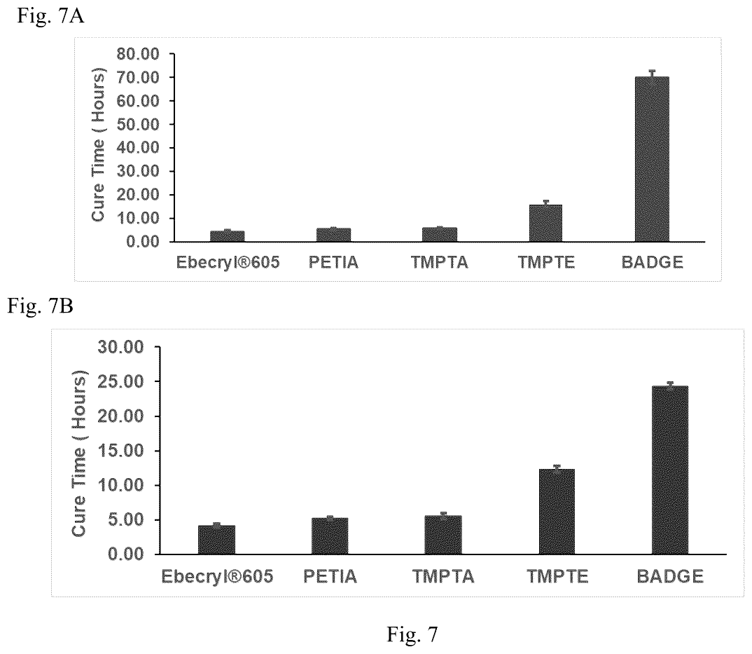

It was studied how long different coating formulas would take to cure using several resin combinations and two sources of ammonia The cure time of thiol to different acrylates and epoxies that were cured by 28-30% aqueous solution of ammonia showed that the epoxy acrylate, Ebecryl®605-TT1 formulation takes the least amount of time to cure and on the other hand, the epoxy-thiol (BADGE-TT1) formulation takes around five times more time than acrylate-thiol coatings (FIG. 7).

It is evident that the curing time of thiol-acrylate coatings catalyzed by in situ ammonia from urea-urease reaction is a bit longer than the time taken by the 28-30% ammonium hydroxide solution. The curing time for in situ ammonia catalyzed epoxy-thiol coatings takes three times more time than 28-30% ammonia solution catalyst. The order of coatings in terms of cure time for both curing agents is Ebecryl®605-TT1<PETIA-TT1<TMPTA-TT1<TMPTE-TT1<BADGE-TT1

The decreased curing time could be explained by the perspective of having a number of free hydroxyl groups at the resin's backbone and bond-breaking energy. Ebecryl®605 resin contains two free hydroxyl groups that can increase hydrogen bonding and crosslinking and thus decrease the curing time of coatings. PETIA has one pendant —OH group but TMPTA has no such —OH group in its structure. On the other hand, the diepoxy compound (BADGE) has to spend more energy breaking the C—O sigma bond than the acrylic C═C pi-bond to form a new S—C polymeric bond with the trithiol (TT1). Furthermore, when functionality rises, crosslinking increases as well, hastening the curing process. For example, curing TMPTE-TT1 is faster than BADGE-TT1.

### Mechanical Properties

Scratch and scrape testing was performed to compare mechanical strength among cured coatings. From this mechanical testing, it was found that fillers contribute to increasing mechanical strength.

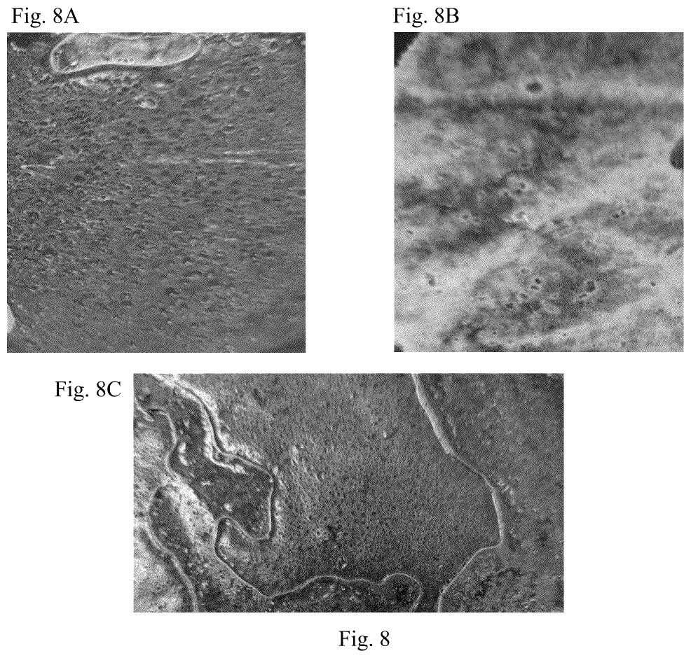

Three different coating samples after curing on a metal substrate showed that cured coatings without fillers are mechanically weaker than coatings with only fume silica, whereas the most mechanically strong cured coatings were made of milled carbon fiber, and fumed silica (FIG. 8). Fumed silica and carbon fiber, respectively, contribute to raising the viscosity and tensile strength of coatings.

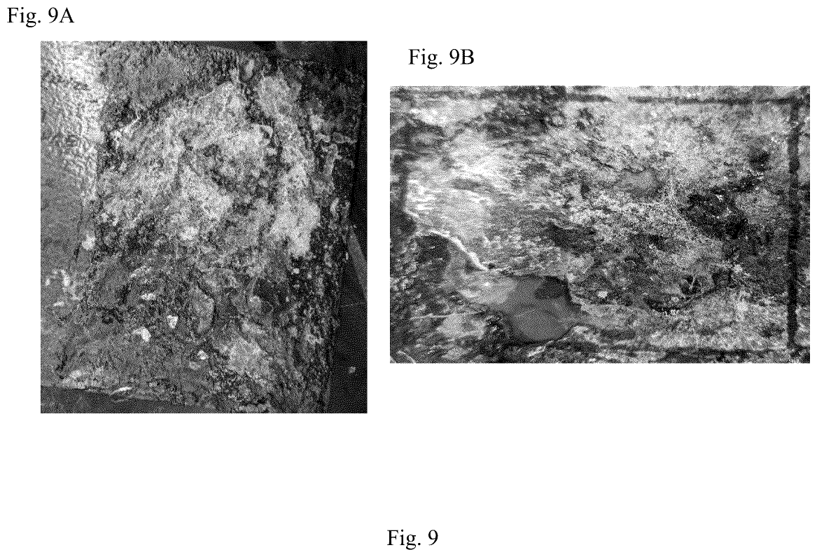

After clearing scratch and scrape testing, thiol-epoxy BADGE-TT1 coatings underwent impact testing, which validated their mechanical strength. TMPTE-TT1 failed scratch and scrape tests with a chisel. On the other hand, cured thiol-acrylate coating compositions are mechanically weak, as all the thiol-acrylate coatings were scratched and scraped with a small chisel, and coatings failed those tests (FIG. 9).

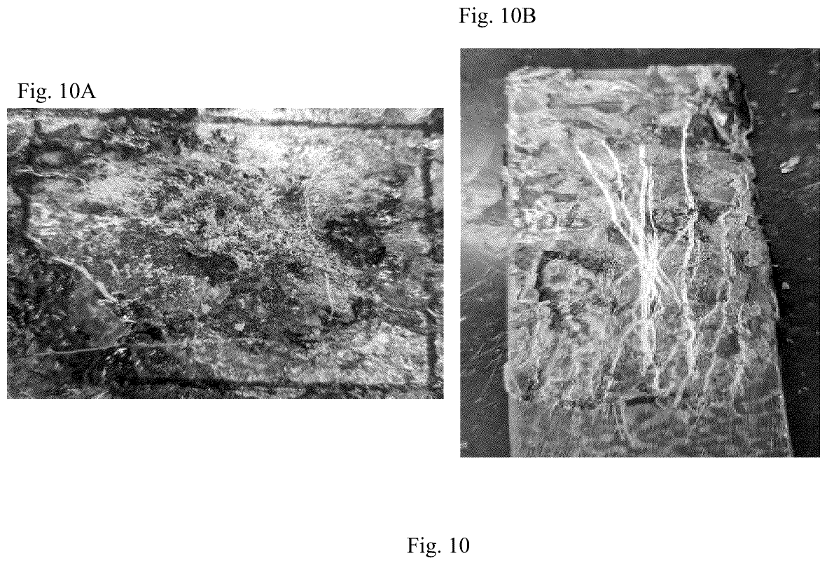

A spotless picture of BADGE-TT1 coating before impact test demonstrated a spotted coating after impacted (FIG. 10). After dropping a 41b weight on the BADGE-TT1 coating 25 times, three nearby impacted links were counted, and the impact score was calculated to be 92.5%, indicating that this coating is mechanically robust.

### Qualitative Chemical Resistance Test

The coatings did not exhibit any softening or delamination when they were immersed in the different chemicals. There were no variations in these qualities between the places that were flooded and those that weren't. Table 3 illustrates the outcomes using various chemicals; the coatings are chemical resistant.

TABLE 2
Results of Qualitative Chemical Resistance
Testing of All Coating Samples

| Chemical | Submerged Time | | Result |
|---|---|---|---|
| Ethanol | 24 | hours | passed |
| Deicing- | 24 | hours | passed |
| defrosting Fluid | | | |
| Motor Oil | 4 | weeks | passed |
| Natural Seawater | 4 | weeks | passed |

### Pot Life Study

TABLE 3
Pot lives of Thiol-Acrylate
and Thiol-Epoxy formulations
Pot life without stabilizers

| Formulations | | |
|---|---|---|
| containing | | |
| FS, CF, & Al₂O₃ | Pot Life | |
| TMPTA-TT1 | <10 | minutes |
| PETIA-TT1 | <4 | hours |
| Ebecryl ®605-TT1 | <2 | hours |
| BADGE-TT1 | <20 | days |
| TMPTE-TT1 | <20 | days |

Pot life after adding stabilizers

| Formulations | | | |
|---|---|---|---|
| containing FS, | | | |
| CF, & Al₂O₃ | Stabilizers | Pot life | |
| TMPTA-TT1 | 1 phr MeHQ & | >400 | days |
| | 0.5 phr PPA | | |
| PETIA-TT1 | 1.5 phr MeHQ & | >400 | days |
| | 0.5 phr PPA | | |
| Ebecryl ®605-TT1 | 1.5 phr MeHQ & | >250 | days |
| | 0.5 phr PPA | | |
| BADGE-TT1 | 1 phr PPA | <30 | days |
| TMPTE-TT1 | 1 phr PPA | <30 | days |

Thiol-enes have limited shelf-life stability due to various factors, including the decomposition of peroxide impurities, the reaction of hydroperoxide impurities to form thiyl radical intermediates that initiate polymerization, the addition of thiol to the ene double bond via base-catalyzed nucleophilic addition, or spontaneous polymerization of the thiol and ene components occurs through the production of radicals, resulting in a ground-state charge transfer complex (Klemm et al., 1993, Angewandte Makromolecular Chemistry, 212, 121; Kuhne et al., 1996, Angewandte Makromolecular Chemistry, 1996, 242, 139). Esfandiary et al. found an excellent result for ideal storage conditions for thiol-enes employing the buffering capabilities of phosphonic acids and the radical stabilizer 4-methoxyphenol (Esfandiari et al., 2013, Journal of Polymer Science Part A: Polymer Chemistry, 51, 4261). For thiol-epoxy pot life only phosphonic acid was used to suppress the pH of thiol from going up because the acid dissociation constant (pKₐ) of thiol is important for its reactivity. Compared to thiols with higher pKa values, the thiol with a pKa of 6.4 is more reactive because it is projected to be about 90% in the thiolate state at pH 7.4. Thiolate is more highly reactive than thiol (Wall et al., 2012, Frontiers in Physiology, 3, 369). The pot life has been increased significantly after adding stabilizers to the formulations (Tables 3a and 3b). Acrylates are showing great promise in extending pot life after the addition of stabilizers. Only PPA was employed to extend the pot life of thiol-epoxy formulations, resulting in a pot life of one month instead of twenty days. Because the Ebecryl®605 structure has two —OH groups on its primary backbone, the pot life of the Ebecryl®605-TT1 system was found to be lower than that of other thiol-acrylate systems. One possible explanation is that the —OH group greatly affects pot life by progressively increasing viscosity as a result of hydrogen bond formation.

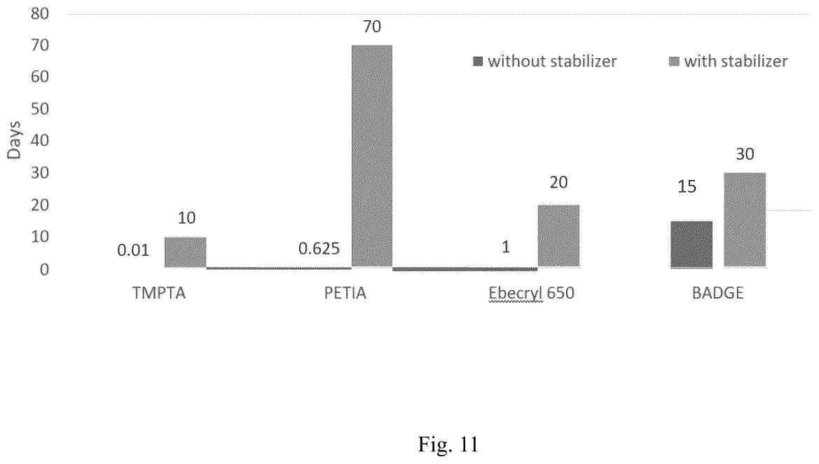

The pot life of the thiol-acrylate system is much less than that of the thiol-epoxy system before adding any stabilizer. Pot life of thiol-epoxy and thiol-acrylate mixture was deemed as one of the prime challenges while applying these coatings on a substrate that's why a millimolar level of stabilizer-4-methoxy phenol and co-stabilizer, phenyl phosphonic acid were added to every formulation which subsequently help increase the pot life (Esfandara et al., 2013, J. Polym. Sci. A: Polym. Chem., 51, 4261). (FIG. 11). This pot life study provides a solid indicator of the pot life expectancy, but is a time-limited study.

Ultimately, a novel approach to cure-on-demand thiol-acrylate and thiol-epoxy coatings using ammonia (NH₃) as the catalyst is presented herein. Mixtures of a trithiol and a triacrylate and fillers were applied to metal surfaces. The Michael addition reaction of the thiol to the acrylate can be catalyzed by base. In the present disclosure, the curing was activated by a 30% aqueous solution of ammonia applied to the resin surface. The loss of ammonia was prevented by a layer of aluminum foil. As the ammonia gas diffuses into the layer, it deprotonates the thiol, allowing addition to the acrylate or epoxy. The thiol-acrylate systems cured in less than 24 hours while the epoxy systems required more time. However, the pot lives of thiol-epoxy systems were longer. To increase the pot life, several weak acids had been tried in millimolar level. Pot lives of months have been achieved. The effects of fillers such as fumed silica and milled carbon fiber were studied. Urease from watermelon seeds was used to catalyze the production of ammonia from an acidic solution of urea with a time delay dependent on the initial pH, which allows covering the layer before ammonia was produced; the amount of ammonia lost to the atmosphere was thus reduced.

Aqueous ammonia and ammonia produced in situ both worked as catalysts for cure-on-demand thiol-epoxy/thiol-acrylate coating curing agent. Short pot lives of the coating formulations have also successfully been overcome through adding some weak acids as stabilizer. Thiol-epoxy coatings are mechanically stronger than thiol-acrylate systems. The use of fillers strengthened the coating mechanically and shortened the curing period. The pot life stabilization and the subsequent curing of resins through step growth fashion was done successfully by using ammonia gas for the first time. This can be called a high solid thermoset polymer synthesis because the work relates to one pot formation without the use of any solvent. The utilization of watermelon seed powder as a source of urease enzyme to produce ammonia into the system can be environmentally and user friendly opposing the direct use of ammonia. Another notable advantage is that the reaction time can be controlled utilizing the autocatalytic mechanism of the urea urease process. The disadvantage is the softness of thiol-acrylate coatings, which could be improved in future investigations.

The disclosures of each and every patent, patent application, and publication cited herein are hereby incorporated herein by reference in their entirety. While present disclosure has been disclosed with reference to specific embodiments, it is apparent that other embodiments and variations of present disclosure may be devised by others skilled in the art without departing from the true spirit and scope of the present disclosure. The appended claims are intended to be construed to include all such embodiments and equivalent variations.

What is claimed is:

1. A method of depositing a coating over a substrate, the method comprising the steps of:
providing a resin composition;
applying the resin composition over a substrate;
exposing the resin composition to a solution comprising a base catalyst; and
polymerizing the resin composition;
wherein the resin composition comprises:
a monomer comprising at least one reactive functionality, wherein the reactive functionality is an alkenyl group or a heterocyclic group;
an initiator comprising at least one thiol functionality, wherein the initiator is capable of being activated using a base catalyst;
wherein the resin composition further comprises:
about 0.1-10 phr silica;
about 5-50 phr milled carbon fiber; and
about 0.1-5 phr of at least one source of urease.

2. The method of claim 1, wherein the solution comprising a base catalyst comprises at least one compound selected from the group consisting of: ammonia, urea, substituted or unsubstituted amine, substituted or unsubstituted hydroxylamine, and a combination thereof.

3. The method of claim 1, wherein the at least one source of urease is selected from the group consisting of: watermelon seeds, jack beans, pea seeds, soybeans, sword beans, ruminal urease, and synthetic urease.

4. The method of claim 1, wherein the solution comprising a base catalyst is about 5% to about 30% base.

5. The method of claim 1, wherein the resin composition is exposed to the solution comprising a base catalyst for about 5 minutes to about 60 hours.

6. The method of claim 1, wherein the solution comprising a base catalyst comprises urea.

7. The method of claim 1, wherein the solution comprising a base catalyst is about 5% to about 20% urea.

8. The method of claim 1, wherein the solution comprising a base catalyst is further acidified.

9. A resin composition comprising:
a monomer comprising at least one reactive functionality, wherein the reactive functionality is an alkenyl group or a heterocyclic group;
an initiator comprising at least one thiol functionality, wherein the initiator is capable of being activated using a base catalyst;
wherein the resin composition further comprises:
about 0.1-10 phr silica;
about 5-50 phr milled carbon fiber; and
about 0.1-5 phr of at least one source of urease.

10. The composition of claim 9, wherein the monomer further comprises a moiety selected from the group consisting of: bisphenol, glycol, alcohol, quaternary carbon, derivatives thereof, and a combination thereof.

11. The composition of claim 9, wherein the reactive functionality is selected from the group consisting of: a methacrylate group, a malonate group, a maleimide group, a vinyl group, an acrylate, an epoxide, an oxetane, and an aziridine.

12. The composition of claim 9, wherein the monomer is selected from the group consisting of: an acrylate, a diacrylate, a triacrylate, an epoxy acrylate, an epoxy diacrylate, an epoxy triacrylate, a glycidyl ether, a diglycidyl ether, and a triglycidyl ether, and a combination thereof.

13. The composition of claim 9, wherein the initiator comprises at least two thiol functionalities.

14. The composition of claim 9, wherein the initiator comprises a compound selected from the group consisting of: trimethylolpropane tris(3-mercaptopropionate) (TT1), trimethylolpropane tris (3-mercaptoacetate), glycol di-3-mercaptopropionate, polypropylene glycol bis (3-mercaptopropionate), ethoxylated trimethylolpropane tris (3-mercaptopropionate), glycol dimercaptoacetate, ethoxylated glycol dimercaptoacetate, 1, 4-bis(3-mercaptobutyryloxy) butane, tris [2-(3-mercaptopropionyloxy) ethyl]isocyanurate, 1,3,5-tris (3-mercaptobutoxyethyl-1,3,5-triazine-2,4,6(1H,3H,5H)-trione, pentaerythritol tetrakis(3-mercaptopropionate), pentaerythritol tetrakis(2-mercaptoacetate), 1,6-hexanedithiol, 1,3-propanedithiol, 1,2-ethanedithiol, polyethylene glycol dithiol comprising 1-10 ethylene glycol repeating units, and a combination thereof.

15. The composition of claim 9, wherein the molar ratio of monomer to initiator is about 1:1.

16. The composition of claim 9, wherein the silica comprises fumed silica, precipitated silica, silica sol, silica gel, or pyrogenic silica.

17. The composition of claim 9, wherein the at least one source of urease is selected from the group consisting of watermelon seeds, jack beans, pea seeds, soybeans, sword beans, ruminal urease, and synthetic urease.

18. The composition of claim 9, wherein the solution comprising a base catalyst comprises urea.

19. A polymerized coating disposed over a substrate, the coating comprising:
a repeat unit formed from a monomer comprising at least one reactive functionality, wherein the reactive functionality is an alkenyl group or a heterocyclic group;
a cross-linker formed from an initiator comprising at least one thiol functionality;
wherein the polymerized coating further comprises:
about 0.1-10 phr silica;
about 5-50 phr milled carbon fiber;
about 0.1-5 phr of at least one source of urease;
about 0.1-5 phr phenyl phosphonic acid; and
about 0.1-5 phr substituted or unsubstituted phenol.

20. The polymerized coating of claim 19, wherein the at least one source of urease is selected from the group consisting of watermelon seeds, jack beans, pea seeds, soybeans, sword beans, ruminal urease, and synthetic urease.
